SEQUENCE LISTING (77 sequences)

SEQ ID NO: 1 - DNA, 20 bp, synthetic construct
  source 1..20: mol_type = other DNA
gggcaggaagcagcagcaca

SEQ ID NO: 2 - DNA, 20 bp, Homo sapiens
  source 1..20: mol_type = genomic DNA
tgtgctgctgcttcctgccc

SEQ ID NO: 3 - DNA, 20 bp, synthetic construct
  source 1..20: mol_type = other DNA
agcagccgcagggcaagcgc

SEQ ID NO: 4 - DNA, 20 bp, Homo sapiens
  source 1..20: mol_type = genomic DNA
gcgcttgccctgcggctgct

SEQ ID NO: 5 - DNA, 20 bp, synthetic construct
  source 1..20: mol_type = other DNA
agcgttagttagccccatgc

SEQ ID NO: 6 - DNA, 20 bp, Homo sapiens
  source 1..20: mol_type = genomic DNA
gcatggggctaactaacgct

SEQ ID NO: 7 - DNA, 20 bp, synthetic construct
  source 1..20: mol_type = other DNA
agcagacactggtgaggaag

SEQ ID NO: 8 - DNA, 20 bp, Homo sapiens
  source 1..20: mol_type = genomic DNA
cttcctcaccagtgtctgct

SEQ ID NO: 9 - DNA, 20 bp, synthetic construct
  source 1..20: mol_type = other DNA
caggagagcagggcgtggga

SEQ ID NO: 10 - DNA, 20 bp, Homo sapiens
  source 1..20: mol_type = genomic DNA
tcccacgccctgctctcctg

SEQ ID NO: 11 - DNA, 20 bp, synthetic construct
  source 1..20: mol_type = other DNA
ggcggcacacacctcttctg

SEQ ID NO: 12 - DNA, 20 bp, Homo sapiens
  source 1..20: mol_type = genomic DNA
cagaagaggtgtgtgccgcc

SEQ ID NO: 13 - DNA, 20 bp, synthetic construct
  source 1..20: mol_type = other DNA
gggtgggggcggcacacacc

SEQ ID NO: 14 - DNA, 20 bp, Homo sapiens
  source 1..20: mol_type = genomic DNA
ggtgtgtgccgcccccaccc

SEQ ID NO: 15 - DNA, 20 bp, synthetic construct
  source 1..20: mol_type = other DNA
ggcaggggtgggggcggcac

SEQ ID NO: 16 - DNA, 20 bp, Homo sapiens
  source 1..20: mol_type = genomic DNA
gtgccgcccccacccctgcc

SEQ ID NO: 17 - DNA, 20 bp, synthetic construct
  source 1..20: mol_type = other DNA
gtaggtggcacagcagaaac

SEQ ID NO: 18 - DNA, 20 bp, Homo sapiens
  source 1..20: mol_type = genomic DNA
gtttctgctgtgccacctac

SEQ ID NO: 19 - DNA, 20 bp, synthetic construct
  source 1..20: mol_type = other DNA
tagcaataggaacaaagcaa

SEQ ID NO: 20 - DNA, 20 bp, Homo sapiens
  source 1..20: mol_type = genomic DNA
ttgctttgttcctattgcta

SEQ ID NO: 21 - DNA, 20 bp, synthetic construct
  source 1..20: mol_type = other DNA
agaaccagcaggcacctgca

SEQ ID NO: 22 - DNA, 20 bp, Homo sapiens
  source 1..20: mol_type = genomic DNA
tgcaggtgcctgctggttct

SEQ ID NO: 23 - DNA, 20 bp, synthetic construct
  source 1..20: mol_type = other DNA
gacccagcggagttcagcag

SEQ ID NO: 24 - DNA, 20 bp, Homo sapiens
  source 1..20: mol_type = genomic DNA
ctgctgaactccgctgggtc

SEQ ID NO: 25 - DNA, 20 bp, synthetic construct
  source 1..20: mol_type = other DNA
taagcaggacccagcggagt

SEQ ID NO: 26 - DNA, 20 bp, Homo sapiens
  source 1..20: mol_type = genomic DNA
actccgctgggtcctgctta

SEQ ID NO: 27 - DNA, 20 bp, synthetic construct
  source 1..20: mol_type = other DNA
gcaaagaccatcagtaagca

SEQ ID NO: 28 - DNA, 20 bp, Homo sapiens
  source 1..20: mol_type = genomic DNA
tgcttactgatggtctttgc

SEQ ID NO: 29 - DNA, 20 bp, synthetic construct
  source 1..20: mol_type = other DNA
ggaaagcactagagcaaaga

SEQ ID NO: 30 - DNA, 20 bp, Homo sapiens
  source 1..20: mol_type = genomic DNA
tctttgctctagtgctttcc

SEQ ID NO: 31 - RNA, 30 nt, synthetic construct
  source 1..30: mol_type = other RNA
ggcggcagtgtgagtaccttcacacgtccc

SEQ ID NO: 32 - DNA, 36 bp, Homo sapiens
  source 1..36: mol_type = genomic DNA
ccgggaggcggcagtgtgagtaccttcacacgtccc

SEQ ID NO: 33 - DNA, 18 bp, Homo sapiens
  source 1..18: mol_type = genomic DNA
gtgtgagtaccttcacac

SEQ ID NO: 34 - DNA, 36 bp, Pan troglodytes; identical to SEQ ID NO: 32
  source 1..36: mol_type = genomic DNA

SEQ ID NO: 35 - DNA, 18 bp, Pan troglodytes
  source 1..18: mol_type = genomic DNA
gtgtgagtaccttcacac

SEQ ID NO: 36 - DNA, 36 bp, Gorilla gorilla; identical to SEQ ID NO: 32
  source 1..36: mol_type = genomic DNA

SEQ ID NO: 37 - DNA, 18 bp, Gorilla gorilla
  source 1..18: mol_type = genomic DNA
gtgtgagtaccttcacac

SEQ ID NO: 38 - DNA, 36 bp, Pongo sp.; identical to SEQ ID NO: 32
  source 1..36: mol_type = genomic DNA

SEQ ID NO: 39 - DNA, 18 bp, Pongo sp.
  source 1..18: mol_type = genomic DNA
gtgtgagtaccttcacac

SEQ ID NO: 40 - DNA, 36 bp, Hylobates sp.; identical to SEQ ID NO: 32
  source 1..36: mol_type = genomic DNA

SEQ ID NO: 41 - DNA, 18 bp, Hylobates sp.
  source 1..18: mol_type = genomic DNA
gtgtgagtaccttcacac

SEQ ID NO: 42 - DNA, 36 bp, Macaca mulatta; identical to SEQ ID NO: 32
  source 1..36: mol_type = genomic DNA

SEQ ID NO: 43 - DNA, 18 bp, Macaca mulatta
  source 1..18: mol_type = genomic DNA
gtgtgagtaccttcacac

SEQ ID NO: 44 - DNA, 36 bp, Macaca fascicularis; identical to SEQ ID NO: 32
  source 1..36: mol_type = genomic DNA

SEQ ID NO: 45 - DNA, 18 bp, Macaca fascicularis
  source 1..18: mol_type = genomic DNA
gtgtgagtaccttcacac

SEQ ID NO: 46 - DNA, 36 bp, Papio sp.; identical to SEQ ID NO: 32
  source 1..36: mol_type = genomic DNA

SEQ ID NO: 47 - DNA, 18 bp, Papio sp.
  source 1..18: mol_type = genomic DNA
gtgtgagtaccttcacac

SEQ ID NO: 48 - DNA, 36 bp, Chlorocebus sabaeus
  source 1..36: mol_type = genomic DNA
ccgggaggcggcagtgtgagtaccttcacacgtcct

SEQ ID NO: 49 - DNA, 18 bp, Chlorocebus sabaeus
  source 1..18: mol_type = genomic DNA
gtgtgagtaccttcacac

SEQ ID NO: 50 - DNA, 36 bp, Callithrix sp.; identical to SEQ ID NO: 32
  source 1..36: mol_type = genomic DNA

SEQ ID NO: 51 - DNA, 18 bp, Callithrix sp.
  source 1..18: mol_type = genomic DNA
gtgtgagtaccttcacac

SEQ ID NO: 52 - DNA, 36 bp, Saimiri sp.; identical to SEQ ID NO: 32
  source 1..36: mol_type = genomic DNA

SEQ ID NO: 53 - DNA, 18 bp, Saimiri sp.
  source 1..18: mol_type = genomic DNA
gtgtgagtaccttcacac

SEQ ID NO: 54 - DNA, 36 bp, Galago sp.
  source 1..36: mol_type = genomic DNA
ccaggaggcggcagtgtgagtaccttcacacgttcc

SEQ ID NO: 55 - DNA, 18 bp, Galago sp.
  source 1..18: mol_type = genomic DNA
gtgtgagtaccttcacac

SEQ ID NO: 56 - DNA, 36 bp, Mus musculus
  source 1..36: mol_type = genomic DNA
ccgggtggaggcagtgtgagtactgtcgcagtctcc

SEQ ID NO: 57 - DNA, 36 bp, Rattus sp.
  source 1..36: mol_type = genomic DNA
ccgggcggaggcagtgtgagtactgtcgcactctcc

SEQ ID NO: 58 - RNA, 29 nt, Homo sapiens
  source 1..29: mol_type = genomic RNA
gcgtccgctcgagcttactgagacactaa

SEQ ID NO: 59 - RNA, 53 nt, Homo sapiens
  source 1..53: mol_type = genomic RNA
atctgttggtttctgctgtgccacctacccaccctgttggtgttgctttgttc

SEQ ID NO: 60 - RNA, 29 nt, Macaca mulatta
  source 1..29: mol_type = genomic RNA
gtgtccgttcgagcttgctgagacattca

SEQ ID NO: 61 - RNA, 53 nt, Macaca mulatta
  source 1..53: mol_type = genomic RNA
atctgttggtttctgctgtgccgcctacccaccctgttgatgatgctttgctc

SEQ ID NO: 62 - RNA, 29 nt, Callithrix sp.
  source 1..29: mol_type = genomic RNA
gcgtccactcaagcttgctgagacgctac

SEQ ID NO: 63 - RNA, 22 nt, Callithrix sp.
  source 1..22: mol_type = genomic RNA
atctgttgatttccactgtgcc

SEQ ID NO: 64 - RNA, 28 nt, Callithrix sp.
  source 1..28: mol_type = genomic RNA
tgtctaccctgttggtgtcactttgctc

SEQ ID NO: 65 - RNA, 29 nt, Saimiri sp.
  source 1..29: mol_type = genomic RNA
gcgtccactcaagcttgctgagacgttac

SEQ ID NO: 66 - RNA, 53 nt, Saimiri sp.
  source 1..53: mol_type = genomic RNA
atgtgttgatttctgctgtgccgcctgcctaccctgttggtgtcgctttgctc

SEQ ID NO: 67 - RNA, 39 nt, Mus musculus
  source 1..39: mol_type = genomic RNA
ctgctcactcatgcttgctgagacacttactgcagctgt

SEQ ID NO: 68 - RNA, 13 nt, Mus musculus
  source 1..13: mol_type = genomic RNA
ggtttctgccgtg

SEQ ID NO: 69 - RNA, 12 nt, Mus musculus
  source 1..12: mol_type = genomic RNA
cgcctgccttct

SEQ ID NO: 70 - RNA, 18 nt, Mus musculus
  source 1..18: mol_type = genomic RNA
gttggtgttgcttcgctc

SEQ ID NO: 71 - RNA, 91 nt, Mus musculus
  source 1..91: mol_type = genomic RNA
tgctcactcatgcttgctgagacacttactgcagctgtggtttctgccgtgcgcctgccttctgttggtgttgcttcgctcttctcagtag

SEQ ID NO: 72 - DNA, 230 bp, Homo sapiens
  source 1..230: mol_type = genomic DNA
tgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcc

SEQ ID NO: 73 - DNA, 177 bp, Homo sapiens
  source 1..177: mol_type = genomic DNA
tgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtc

SEQ ID NO: 74 - RNA, 21 nt, synthetic construct
  source 1..21: mol_type = other RNA
tgctcactcatgcttgctgag

SEQ ID NO: 75 - RNA, 22 nt, synthetic construct
  source 1..22: mol_type = other RNA
acacttactgcagctgtggttt

SEQ ID NO: 76 - RNA, 22 nt, synthetic construct
  source 1..22: mol_type = other RNA
tgccgtgcgcctgccttctgtt

SEQ ID NO: 77 - RNA, 22 nt, synthetic construct
  source 1..22: mol_type = other RNA
ggtgttgcttcgctcttctcag

(19) United States
(12) Patent Application Publication
(10) Pub. No.: US 2025/0084413 A1
(43) Pub. Date: Mar. 13, 2025

(54) METHODS AND COMPOSITIONS TO REGULATE MAPT SPLICING FOR MODELING AND TREATMENT OF TAUOPATHIES

(71) Applicant: THE TRUSTEES OF COLUMBIA UNIVERSITY IN THE CITY OF NEW YORK, New York, NY (US)

(72) Inventors: Yocelyn RECINOS, New York, NY (US); Chaolin ZHANG, Scarsdale, NY (US)

(21) Appl. No.: 18/819,967

(22) Filed: Aug. 29, 2024

Related U.S. Application Data
(63) Continuation of application No. PCT/US2023/063719, filed on Mar. 3, 2023.
(60) Provisional application No. 63/346,786, filed on May 27, 2022.
(60) Provisional application No. 63/316,547, filed on Mar. 4, 2022.

(51) Int. Cl. C12N 15/113; A61P 25/28; C12N 9/22; C12N 15/11

(52) U.S. Cl. CPC C12N 15/113; A61P 25/28; C12N 9/22; C12N 15/11; C12N 2310/11; C12N 2310/20; C12N 2320/33

(57) ABSTRACT

Compounds for, and methods of, modeling and treating a neurodegenerative disease, including without limitation frontal temporal dementia (FTD), are provided. An effective dose of a pharmaceutical composition is administered to a patient in need thereof, where the pharmaceutical composition targets at least one muscleblind-like protein (MBNL) binding site on exon 10 of microtubule-associated protein tau (MAPT) to block MBNL binding thereto. The at least one MBNL binding site may be 2 binding sites. The pharmaceutical composition may include the nuclease-inactive dCas13d in complex, with one or more guide RNAs, and/or antisense oligonucleotides (ASOs).

### CROSS-REFERENCE TO RELATED APPLICATIONS

This application is a continuation of International Application No. PCT/US2023/063719, filed on Mar. 3, 2023, which claims priority to U.S. Provisional Application No. 63/316,547 filed on Mar. 4, 2022, and U.S. Provisional Application No. 63/346,786 filed on May 27, 2022, the entire contents of which applications are incorporated herein by reference thereto.

### GOVERNMENT LICENSE RIGHTS

This invention was made with government support under grant nos. GM136856 and GM145279, awarded by the National Institutes of Health (NIH). The government has certain rights in the invention.

### INCORPORATION-BY-REFERENCE OF MATERIAL ELECTRONICALLY FILED

The official copy of the sequence listing is submitted electronically in ST.26 XML format having the file name “113WO-PCT.xml” created on Nov. 12, 2024, and having a size of 69,414 bytes, and is filed concurrently with the specification. The Sequence Listing ST.26 XML file is part of the specification and is herein incorporated by reference in its entirety.

### TECHNICAL FIELD

The development and administration of pharmaceutical compositions that target at least one muscleblind-like protein (MBNL) binding site near exon 10 of microtubule-associated protein tau (MAPT), to block MBNL binding thereto and modulate exon 10 splicing, provides treatment of neurodegenerative diseases—known collectively as tauopathy—in a patient.

### DESCRIPTION OF THE RELATED ART

Alternative splicing (AS) of precursor messenger RNA is a ubiquitous molecular mechanism to produce multiple transcript and protein isoforms from single genes. AS was proposed to accelerate evolution and divergent AS patterns were found to be widespread across mammals including primates, but mechanisms leading to divergent AS events and their functional impact are poorly understood.

Eukaryotic genes have a split gene structure in which precursor messenger RNA (pre-mRNA), composed of exons and introns, undergoes splicing to produce the correct mRNA for protein translation^{1, 2}. As soon as this discovery was made over four decades ago, it was hypothesized that the split gene structure multiple transcript and protein isoforms to be generated from single genes by alternative splicing (AS), which not only dramatically amplifies the complexity of genetic information encoded in the genome, but also accelerates evolution by contributing to speciation and phenotypic variation among individuals in a population³. This hypothesis is appealing as mammals, including humans, have a similar number of protein-coding genes as compared to lower vertebrates, despite higher organismal complexity. For example, the brain size and function are dramatically expanded in primates, especially in humans when compared to closely related species⁴. At the molecular level, these predictions have been validated. With the advent of deep RNA sequencing (RNA-seq), it is clear that AS is ubiquitous (over 90% of multi-exon genes), and frequently under precise and specific tissue, cell-type, and developmental stage-dependent regulation⁵⁻¹⁰. In addition, many AS events have diverged across mammalian species due to both creation and loss of exons^{11, 12} or quantitative changes in exon inclusion levels^{13, 14} However, the functional implications of these divergent AS events are under debate¹⁵, and it remains poorly understood how changes in splicing-regulatory elements affect their recognition by RNA-binding proteins (RBPs) and lead to a divergence in splicing patterns in different species/lineages.

### SUMMARY OF THE PRESENT TECHNOLOGY

### Lineage-Specific Regulation of Alternative Splicing in the Primate Brain

We modeled inclusion of cassette AS exons in primate brains as a quantitative trait and identified 1,170 exons showing lineage-specific splicing shifts under stabilizing selection. These exons are frequently regulated during development, especially in the human lineage. Among them, we found microtubule binding protein tau (MAPT) exon 10, which is specifically included in the adult brain, shows a two-step evolutionary shift in Catarrhine and hominoid lineages. This shift cannot be explained the previously predicted stem loop structure implicated in dysregulation of exon 10 splicing in frontotemporal dementia (FTD), but is driven by the divergence in developmental stage-specific regulation by Muscleblind (MBNL) through distal intronic binding sites. This suggests AS is a molecular mechanism contributing to evolutionary expansion of brain structure and function in the primates.

We modelled the alternative cassette exon inclusion in the primate brain as a quantitative trait using the Ornstein Uhlenbeck (OU) process to characterize the random drift and selection constraints within the phylogenetic tree. We identified hundreds of exons with lineage-specific splicing shift under stabilized or adaptive selection. Among them, we found a two-step evolutionary change in splicing of the microtubule associated protein tau (MAPT) gene exon 10, which is tightly regulated to maintain a balance of the inclusion (i.e. 4R tau) and skipping (i.e. 3R tau) isoforms in the adult human brain¹⁶⁻¹⁸. Mutations that disrupt this 4R/3R isoform balance can result in the pathological aggregation of tau and the formation of neurofilament tangles (NFT) that cause frontal temporal dementia (FTD)¹⁹⁻²². Further mechanistic dissection identified the divergence of regulatory sequences recognized by muscleblind-like (MBNL) proteins, critical regulators of MAPT exon 10 developmental splicing switch, as a major contributor to the splicing pattern divergence.

### BRIEF DESCRIPTION OF FIGURES

Global Identification and Characterization of Exons with Lineage-Specific Splicing Shifts Under Stabilizing Selection in the Primate Brain.

FIG. 1A. Schematic illustration of testing lineage-specific splicing shift using an Ornstein Uhlenbeck (OU) model.

FIG. 1B. Heatmap showing splicing profiles for exons with lineage-specific splicing shifts in human, hominoid (human+chimp) and Catarrhini (hominoid+old world monkeys) compared to the other primate species. Each exon is a row, and each sample is a column. Exon inclusion level (precent spliced in, Y) is centered by mean across samples. The numbers of significant exons (FDR≤0.1) are indicated on the right.

FIG. 1C. Percentage of exons with lineage-specific splicing shifts showing developmental splicing switches. The percentage among all cassette exons is used for control.

FIG. 1D. Gene ontology (GO) terms associated with exons showing lineage-specific splicing shifts. Genes with exons identified by each branch-specific likelihood ratio (BS-LR) test, or the combined gene list, were used to identify significant GO terms. Only GO terms significant (Benjamini FDR≤0.05) in at least one test are shown and color coded. Grey indicates non-significant GO terms for the respective exon list.

FIG. 1E. Enrichment and deletion of splicing-regulatory elements associated with exons showing lineage-specific increase (left) or decrease (right) in exon inclusion. ESE/ESS: exonic splicing enhancer/silencer, ISE/ISS: intronic splicing enhancer/silencer, EIE/IIE: exonic/intronic identity element. IIE5ss/IIE3ss: IIE associated with 5′ or 3′ splice sites.

FIG. 1F. Concordance of splicing change directions between RNA-seq data and predicted changes in splice site strengths for exons with lineage-specific splicing shifts. Splice site strengths were predicted either by MaxEntScan based on splice site motifs or by SpliceAI based on both local and distal pre-mRNA sequences.

### MAPT Exon10 Splicing Undergoes a Two-Step Evolutionary Splicing Shift in Primates.

FIG. 2A. The genomic locus where the MAPT gene is located and the MAPT gene structure. The major alternative exons 2,3 and 10 are highlighted.

FIG. 2B. A stem loop structure at the border of exon 10 and intron 10 that overlaps with the 5′ splice site that was previously predicted to repress exon 10 inclusion. This stem loop structure is predicted to be destabilized by mutations in FTD patients and in mice, resulting in increase in exon 10 inclusion.

FIG. 2C. The bar plot shows mean+/−standard error. The source of RNA-seq data is indicated in the parentheses, L: Lister et al.²³, N: NHPRTR²⁴, B: Brawand et al.⁶⁴, W: Weyn-Vanhentenryck et al.⁵¹.

FIG. 2D. Multiple alignments of sequences near the 5′ splice site of exon 10 containing the predicted a stem loop structure. Note the perfect conservation of stem loop structure in primates (shaded).

### Divergence in Developmental Stage-Specific Regulation by MBNL Drives Divergence in Splicing Pattern Between Human and Mouse.

FIG. 3A. Cassette exons with conserved, mouse-specific, or human-specific late developmental splicing switches (left). Regulation of these exons by MBNL, as determined by comparison of wild type vs. Mbnl1/2 brains in mice and control and DM1 brains in humans (right). Exons with MBNL-dependent inclusion are indicated by red bars, while exons with MBNL-dependent skipping are indicated by blue bars. The fraction of developmentally regulated exons that are MBNL targets are also indicated below each heatmap.

FIG. 3B. Changes of MAPT exon 10 inclusion in human cortex at different developmental stages (left) and in myotonic dystrophy type 1 (DM1) patient brains (right), as quantified by RNA-seq.

FIG. 3C. Changes of MAPT exon 10 inclusion in mouse cortex at different developmental stages (left) and in Mbnl1/2 double knockout (DKO) brains (right), as quantified by RNA-seq.

FIG. 3D. Exon 10 inclusion in human and mouse MAPT minigenes (hMART and mMART) in control HEK293T cells (pcDNA), cells with MBNL1/2 knockdown using siRNAs (siMBNL1/2) and cells with overexpression of MBNL2 (pMBNL2). Representative images show the confirmation of protein-expression using immunoblots (GAPDH is used for control) and exon 10 inclusion using RT-PCR. Quantification of exon 10 inclusion (mean+/−SEM from n=3 replicates) is shown at the bottom.

### Divergence in Distal MBNL Binding Site in the Downstream Intron Leads to Lineage-Specific Splicing Shift of MAPT Exon 10.

FIG. 4A. MBNL binding sites around MAPT exon 10 as measured by MBNL2 CLIP data and bioinformatically predicted YGCY clusters. The two major binding sites in the downstream intron are highlighted by shaded boxes. Multiple alignments of site 2 sequences in five different primate species analyzed with mMAPT and hMAPT minigene splicing reporters are shown at the bottom.

FIG. 4B,C. MBNL binding site2 deletion (D) or replacement (E) experiments using mMAPT minigene with (pFLAG-MBNL2) or without (pcDNA) MBNL2 overexpression. Representative gel image show confirmation of protein expression using immunoblots (GAPDH is used for control) and exon 10 inclusion using RT-PCR. Quantification of exon 10 inclusion (mean+/−SEM from n=3 replicates) is shown at the bottom.

FIG. 4D,E. MBNL binding site 1 or 2 replacement experiments using hMAPT minigene with (pFLAG-MBNL2) or without (pcDNA) MBNL2 overexpression. Representative gel image show confirmation of protein expression using immunoblots (GAPDH is used for control) and exon 10 inclusion using RT-PCR. Quantification of exon 10 inclusion (mean+/−SEM from n=3 replicates) is shown at the bottom.

Modulation of MAPT Exon 10 Splicing by Steric Hindrance of MBNL Binding Site 2 Using dCas13/gRNAs.

FIG. 5A. Schematic of splicing modulation using dCas13d/gRNA targeting specific sequences in MBNL binding site 2. Four gRNAs overlapping with MBNL-binding YGCY elements were designed and tested.

FIG. 5B. Exon inclusion without or with dCas13d/gRNA targeting using individual gRNAs or all four gRNAs together. Representative gel image show confirmation of protein expression using immunoblots (GAPDH is used for control) and exon 10 inclusion using RT-PCR. Quantification of exon 10 inclusion (mean+/−SEM from n=3 replicates) is shown at the bottom.

### Modulation of MAPT Exon 10 Splicing by Steric Hindrance of MBNL Binding Site Using Antisense Oligonucleotides (ASOs).

FIG. 6A. MBNL binding sites located in the distal intronic regions downstream of MAPT exon 10 were targeted with 20-nt 2′-MOE-PS ASOs. A total of 15 ASOs were designed to cover the MBNL binding motif “YGCY” located at these sites.

FIG. 6B. Representative gel of RT-PCR products for MAPT exon 10 inclusion after transfection of ASO1-15 (80 nM) and human MAPT exon 10 minigene (25 ng) in HEK293T cells. PCR was performed using minigene-specific primers. Note that ASO1 and ASO4 decrease inclusion levels of exon 10 most effectively. Error bars represent standard error of the mean from duplicates.

### Hierarchical Clustering of Cassette Exons Based on Exon Inclusion in Adult Brain Across Primate Species.

The heatmap shows mean-centered exon inclusion level across samples. Six clusters of exons showing lineage-specific splicing shifts are highlighted.

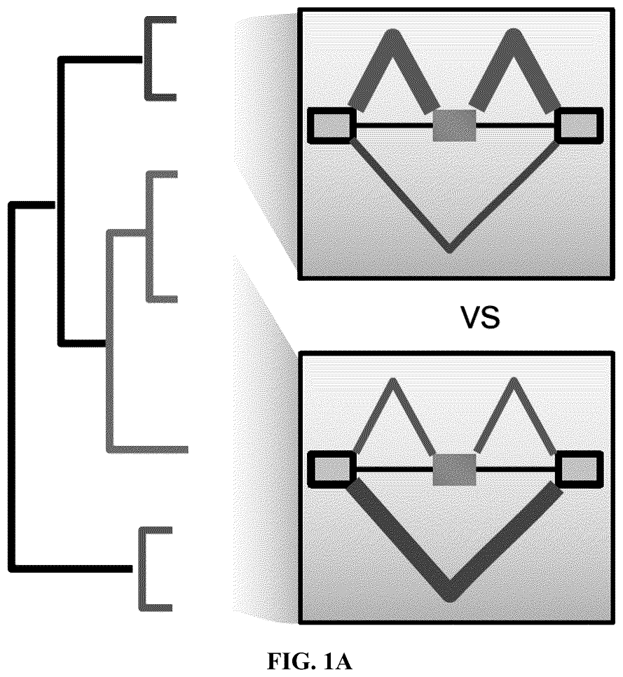

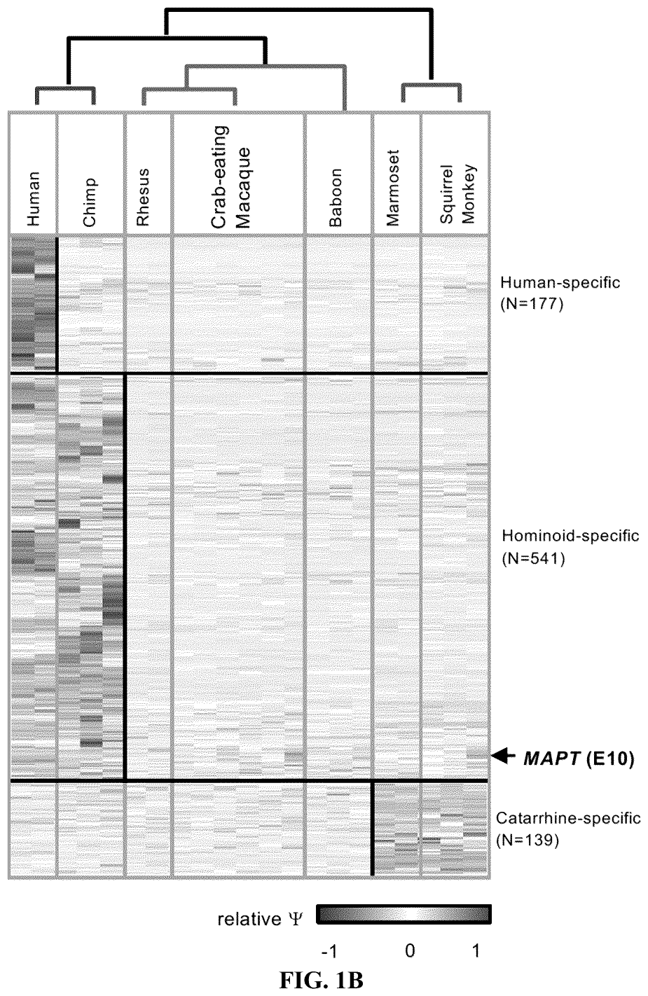

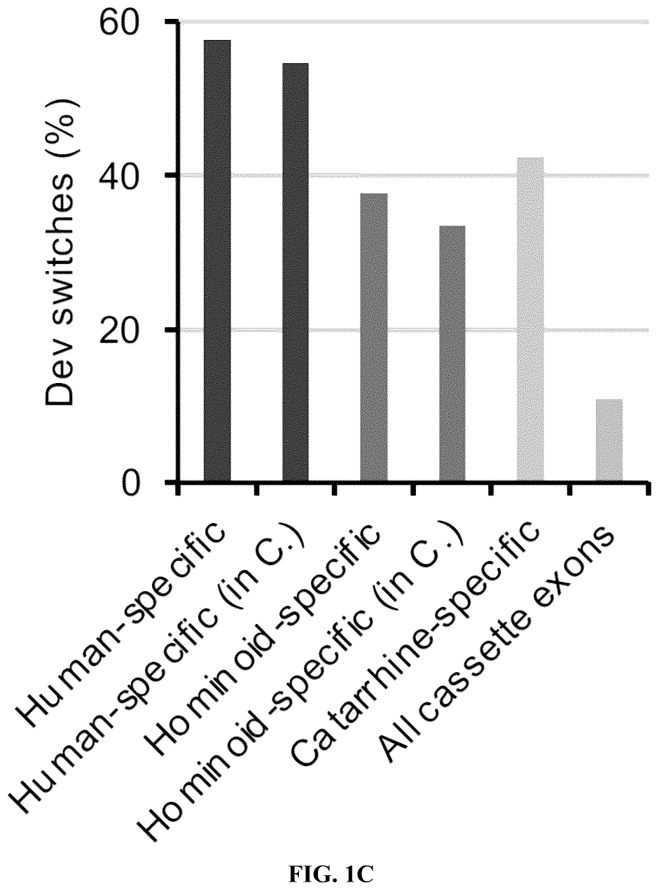

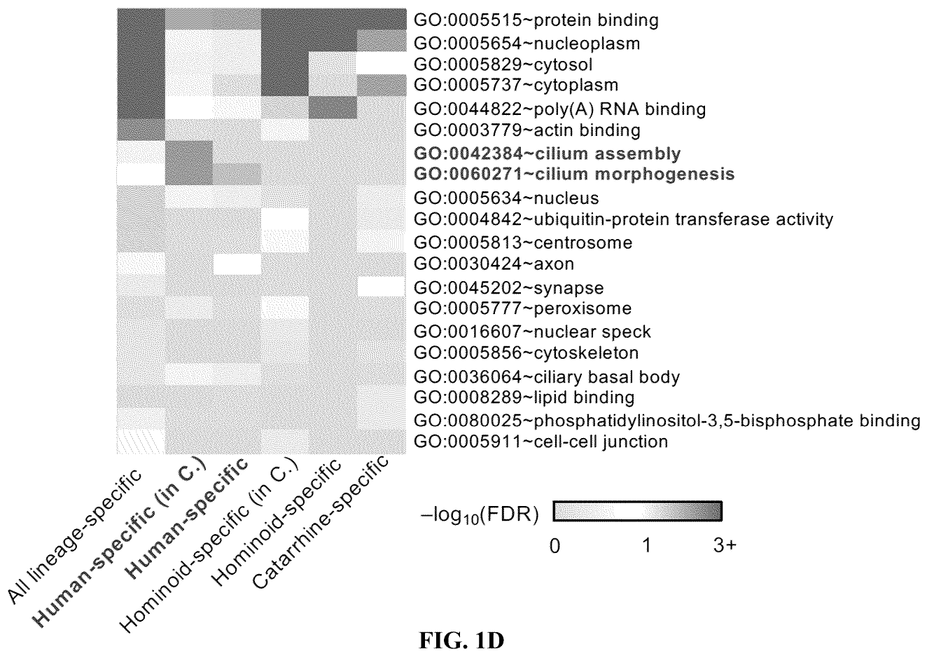

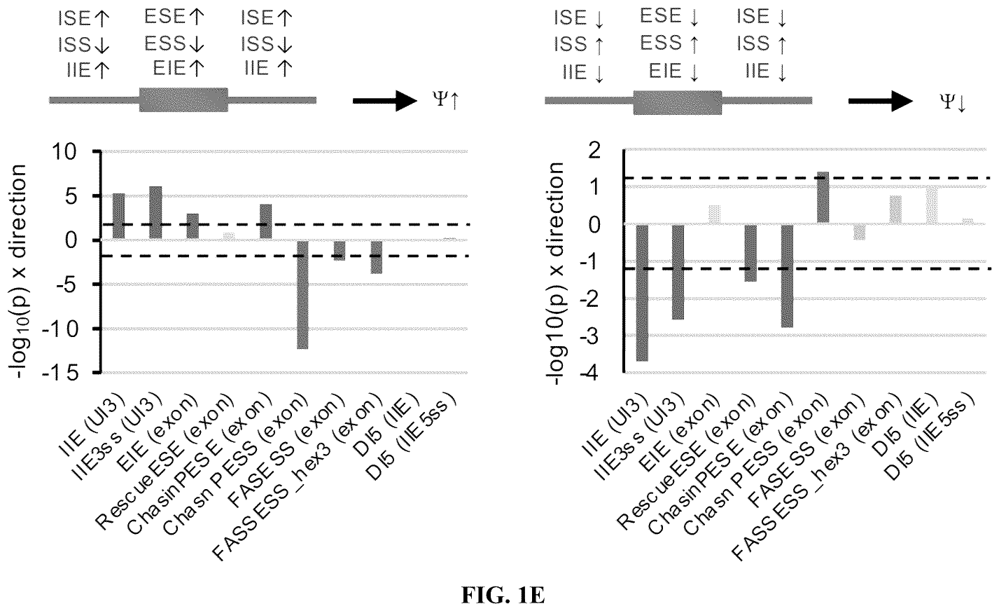

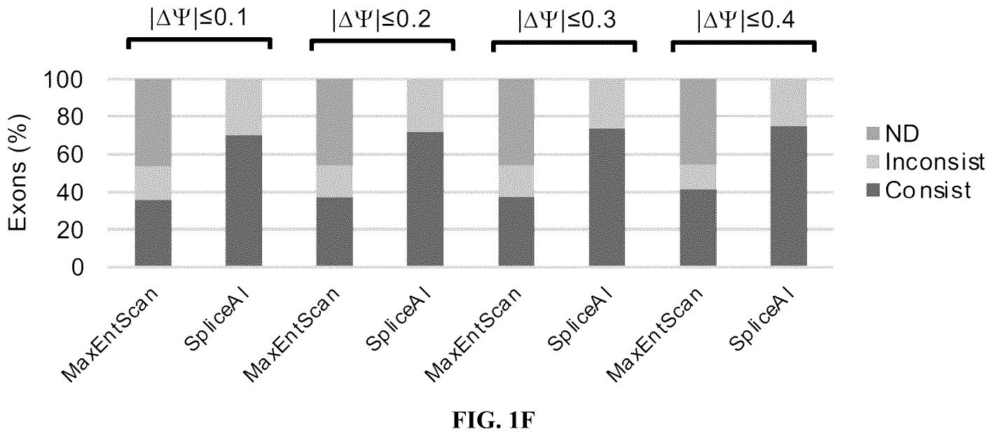

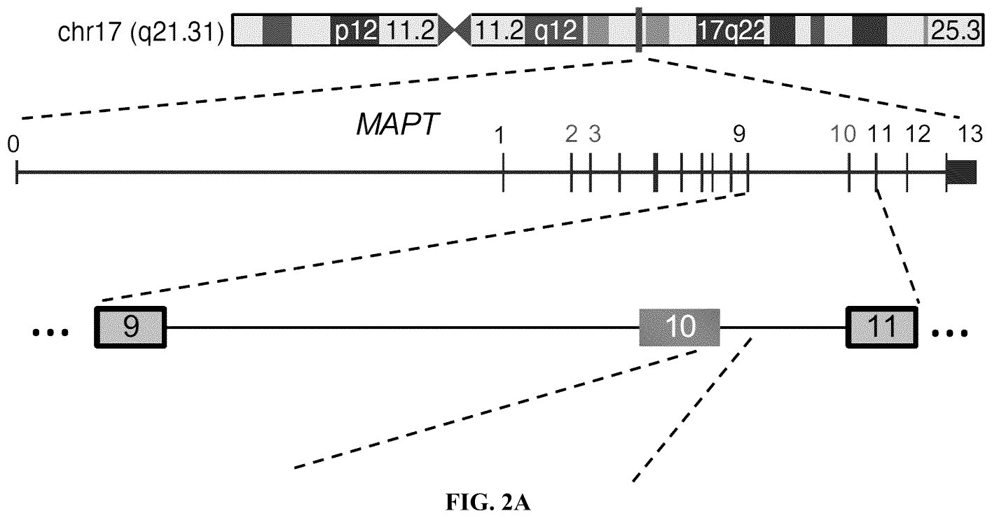

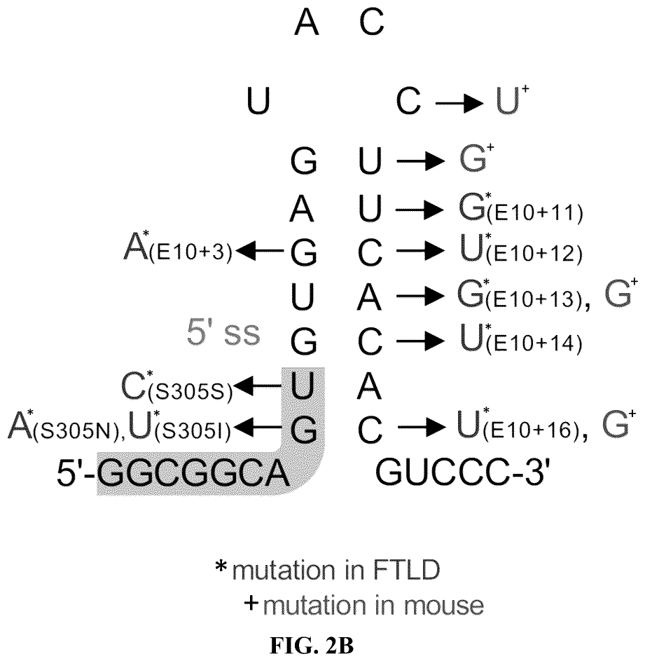

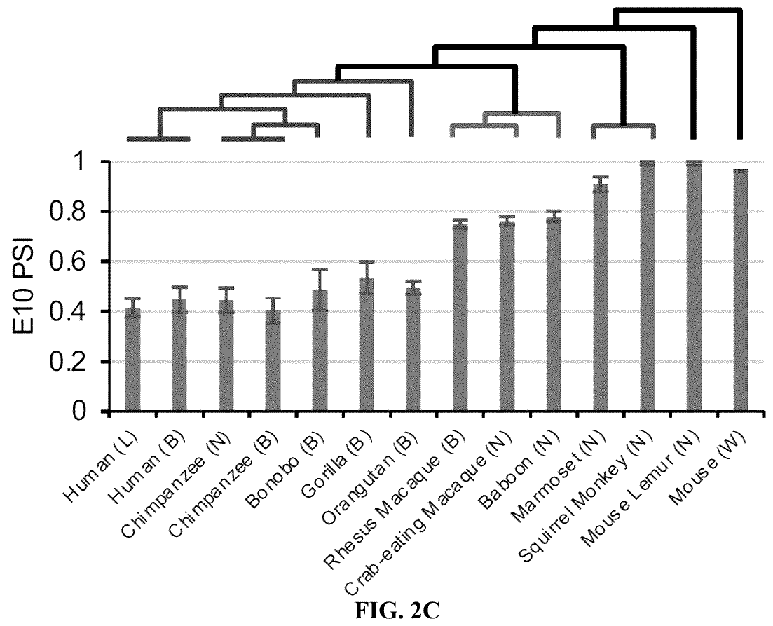

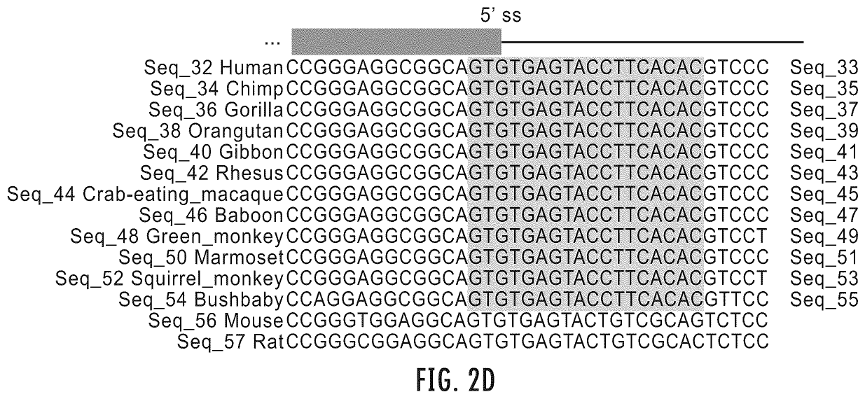

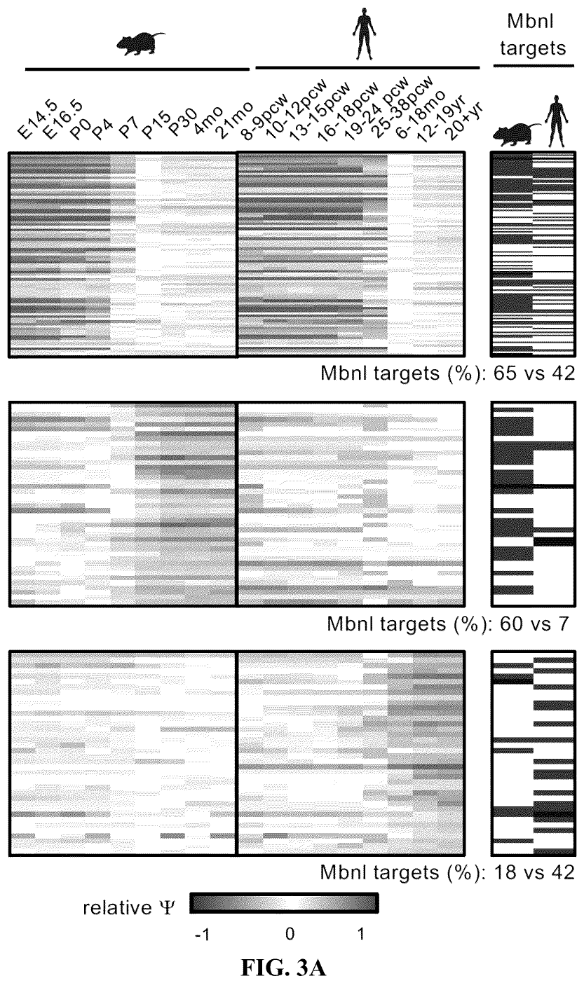

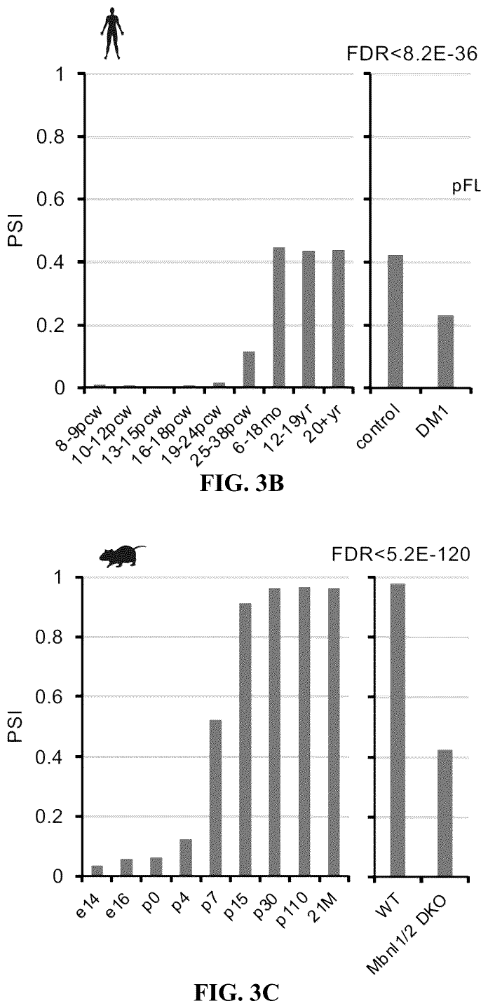

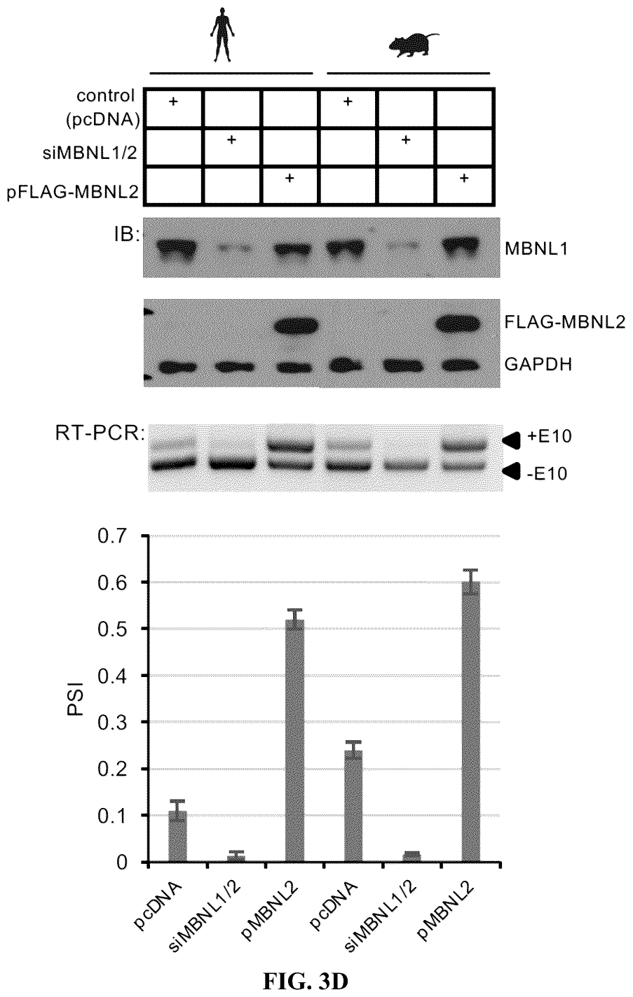

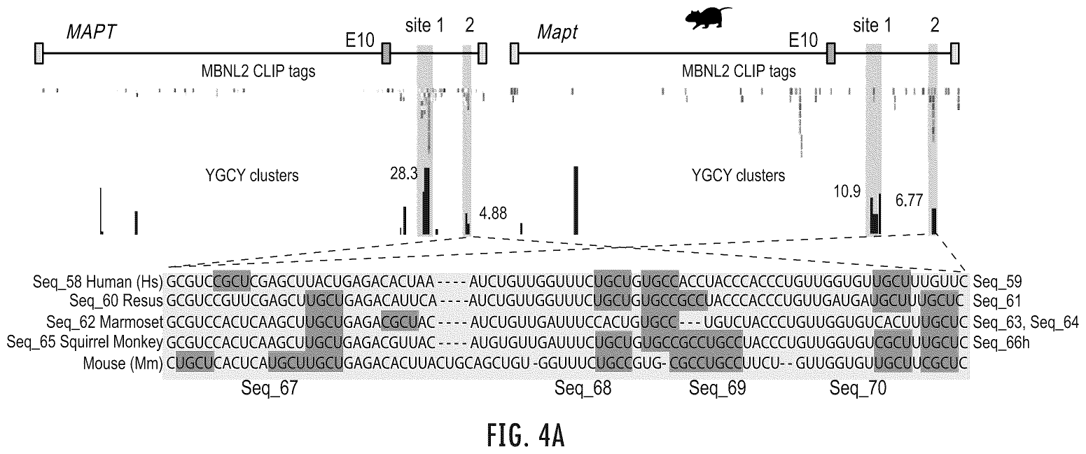

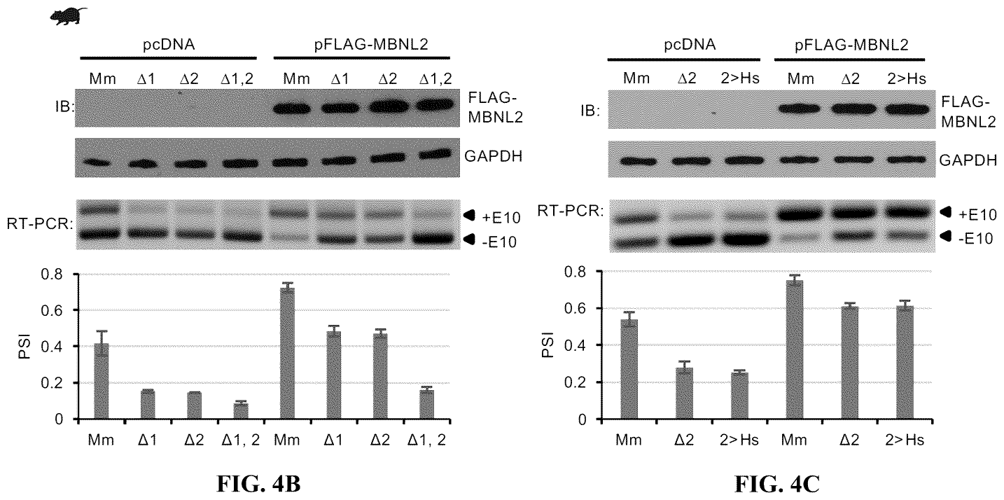

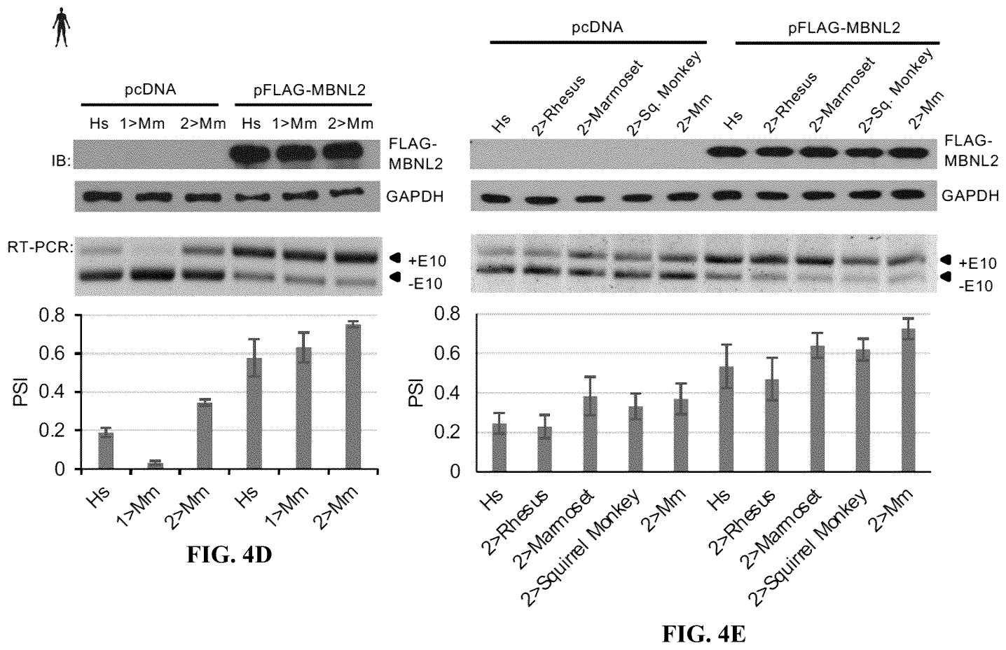

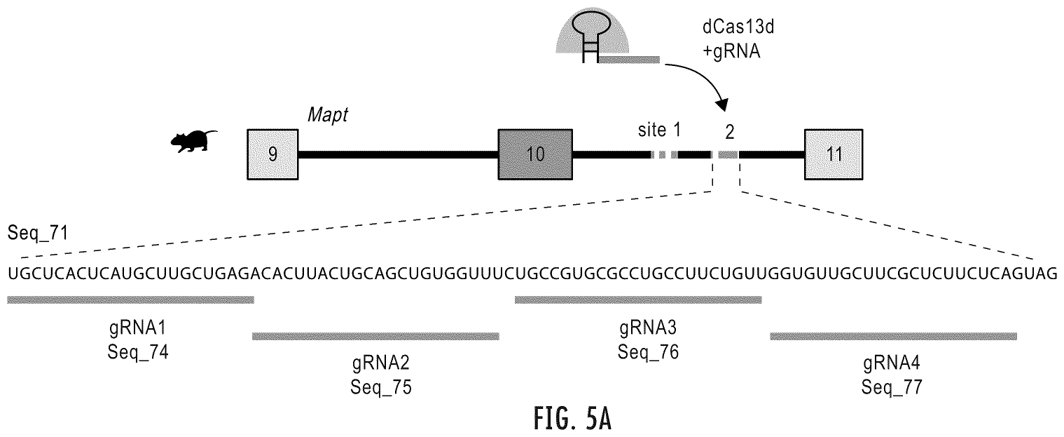

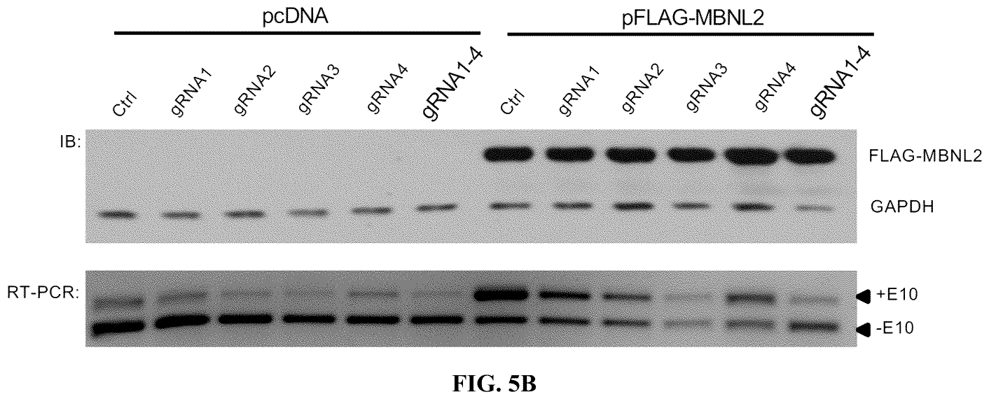

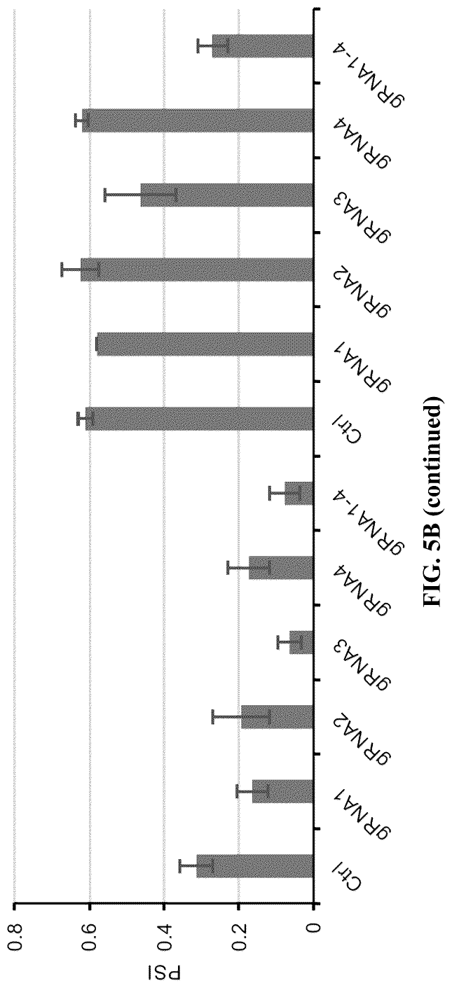

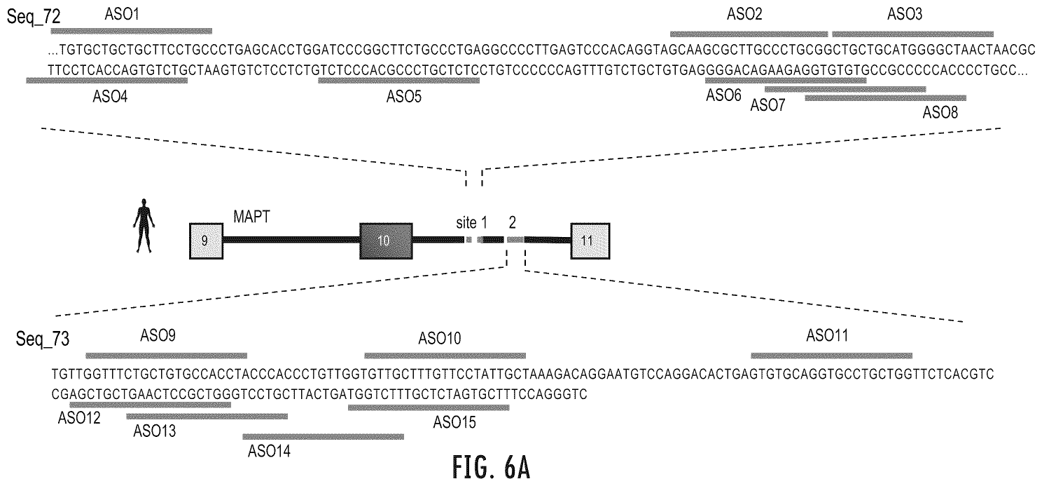

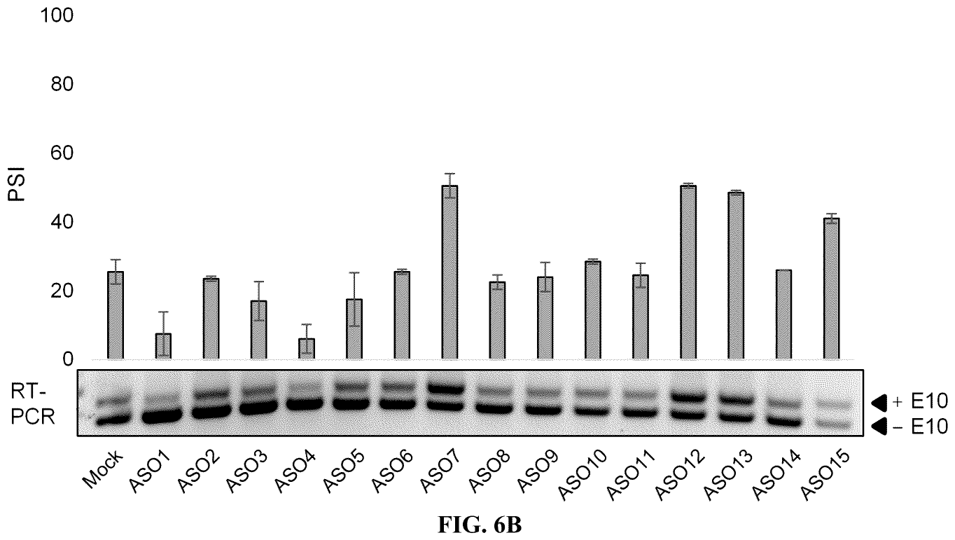

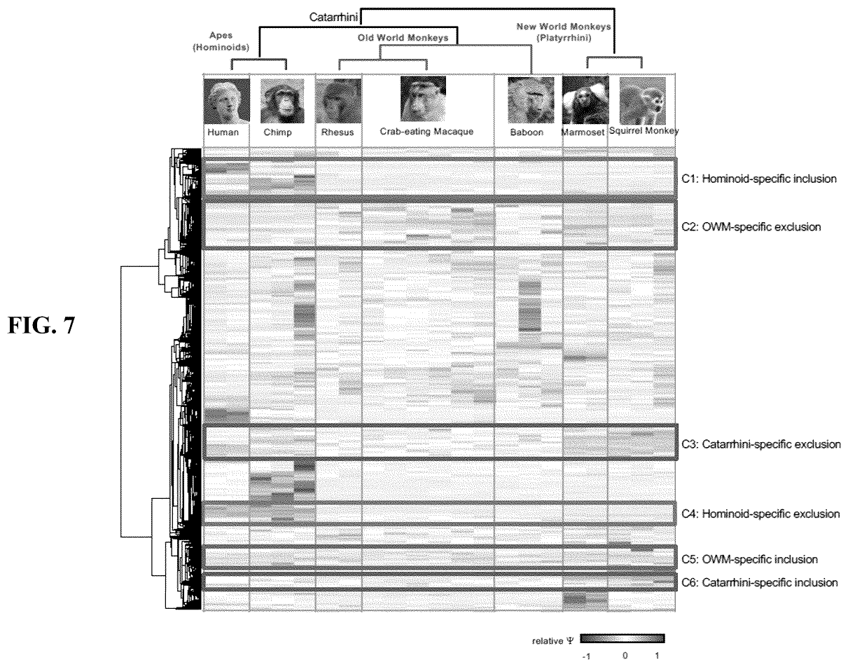

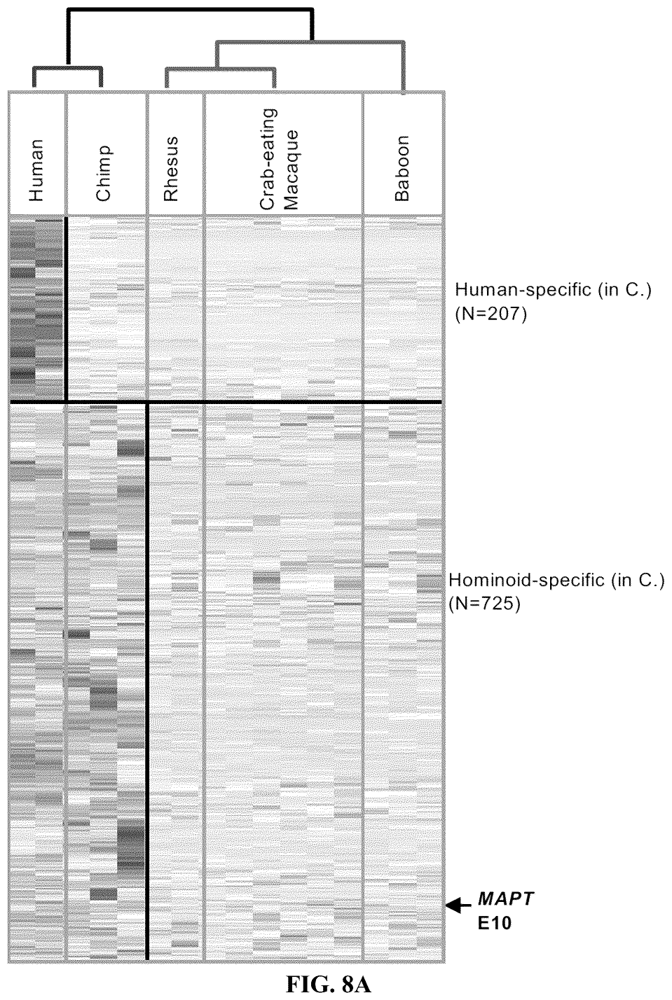

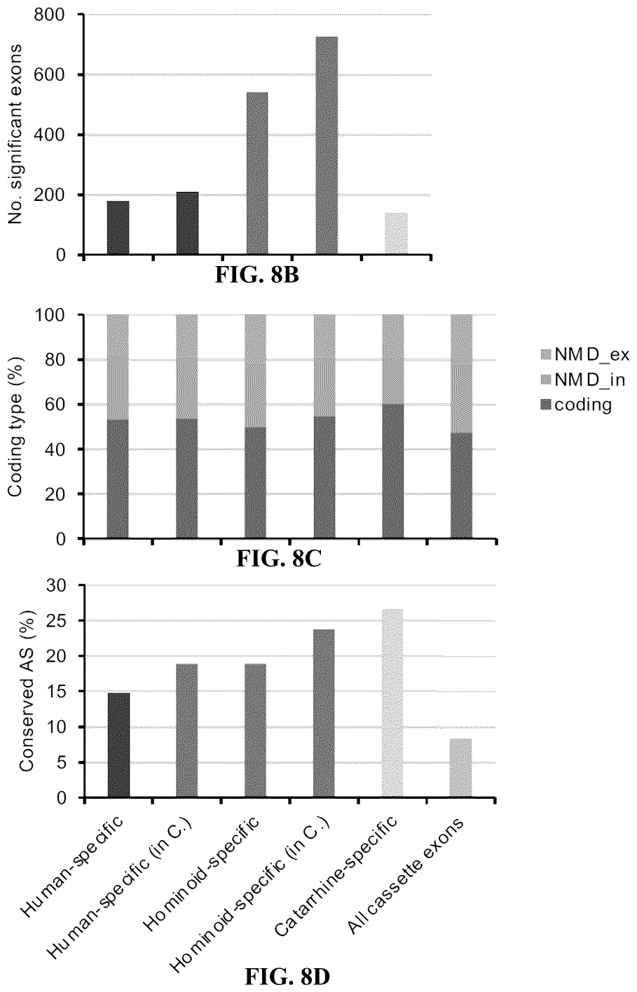

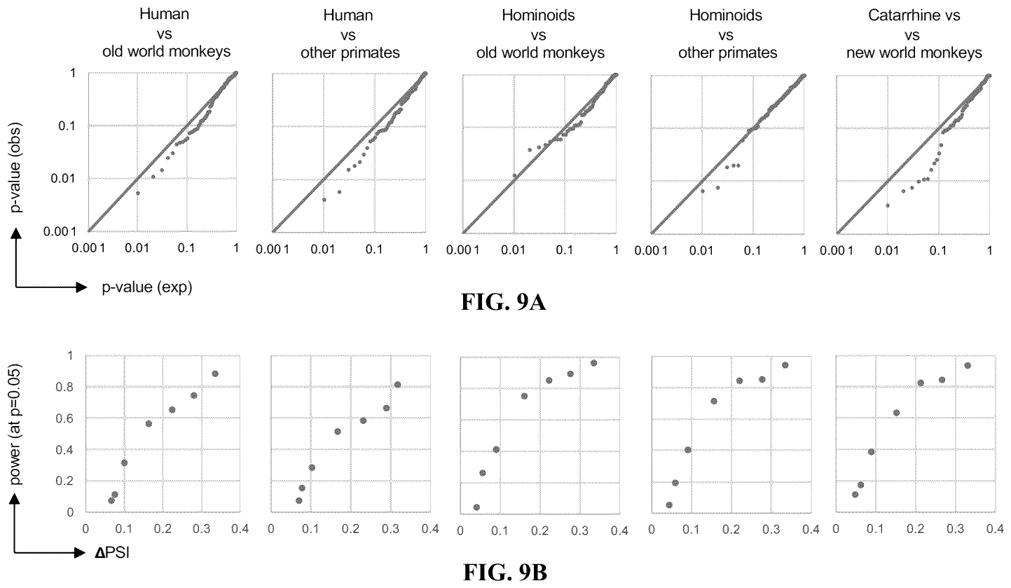

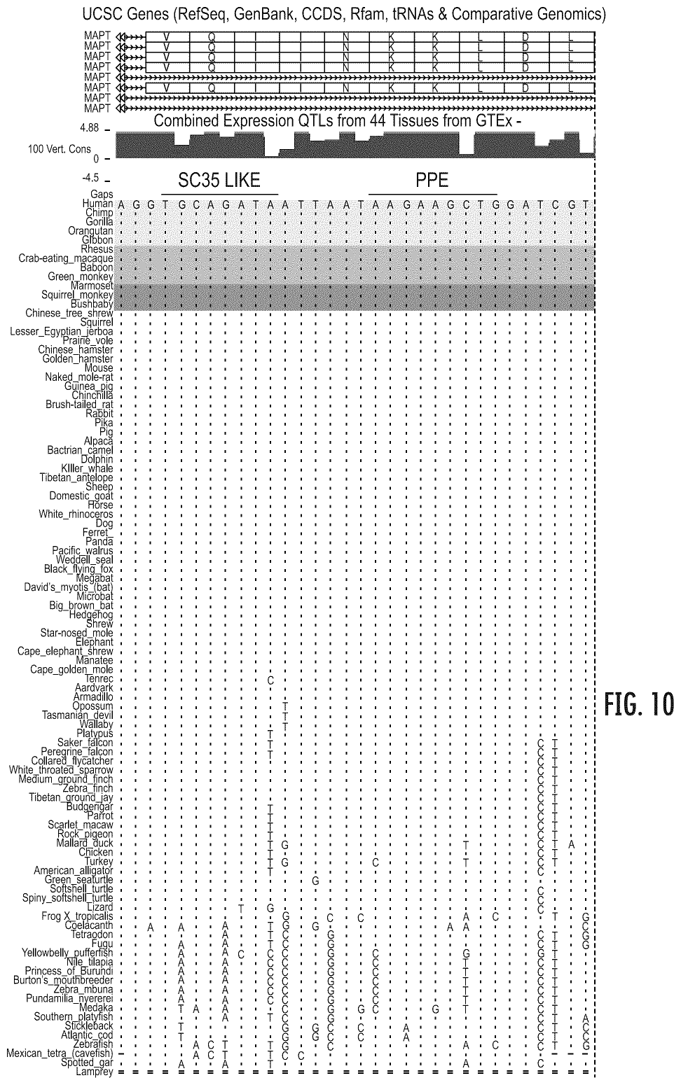

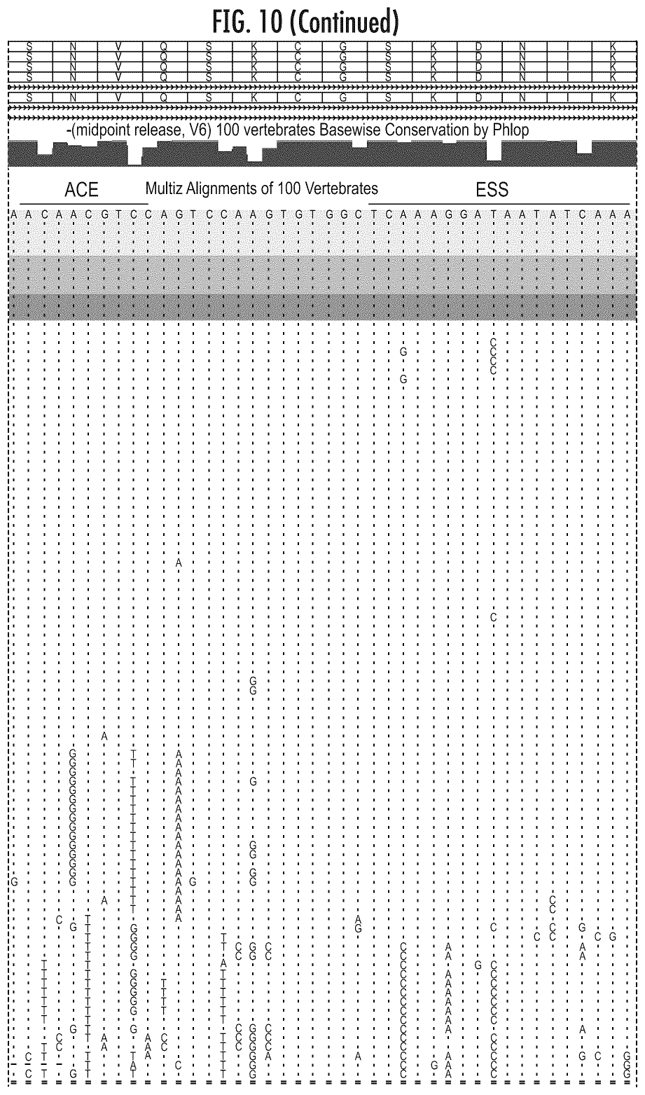

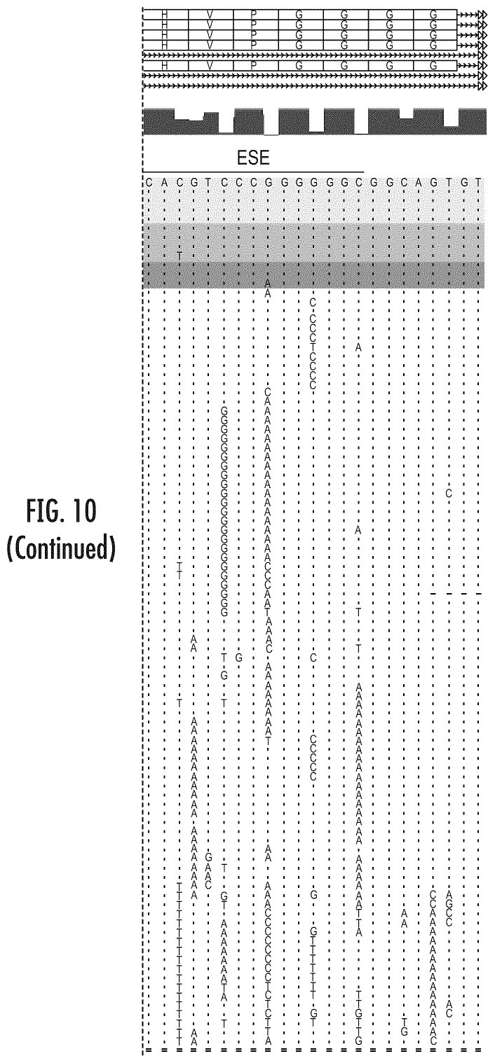

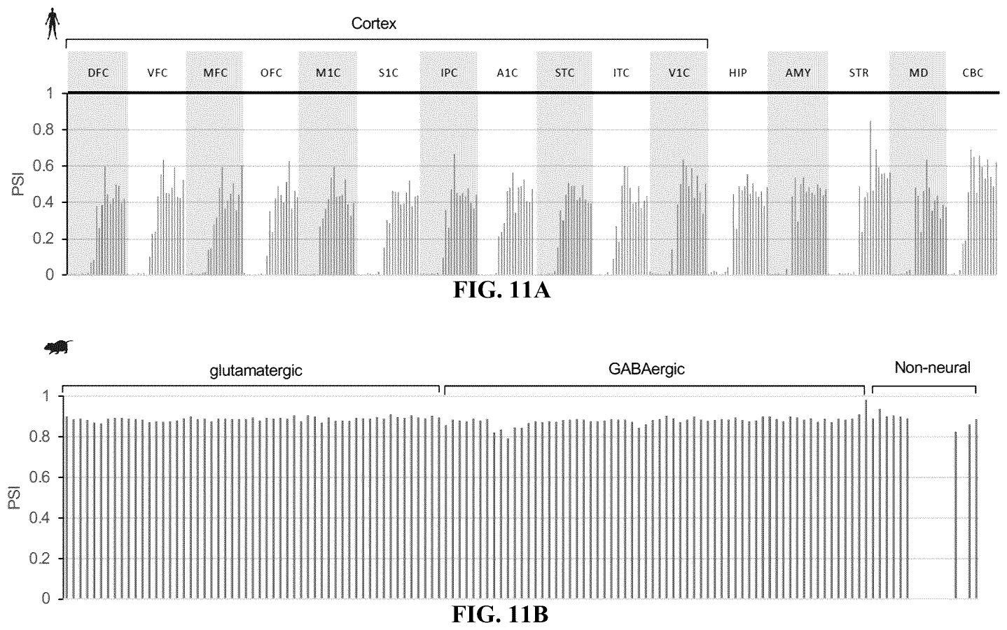

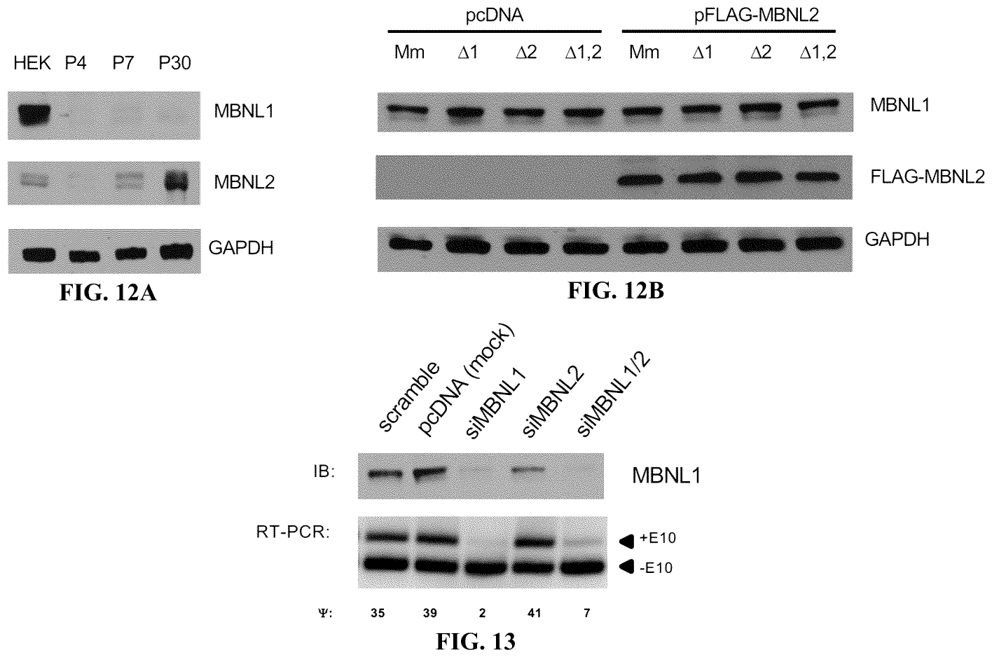

Global Identification of Exons with Lineage Specific Splicing Shift Under Stabilizing Selection. Related to FIG. 1 in the Main Text.

FIG. 8A. Similar to main FIG. 1b, but exons with lineage-specific splicing shifts were identified using 5 Catarrhine (hominoids+old world monkeys). Each exon is a row, and each sample is a column. Exon inclusion level (precent spliced in, Y) is centered by mean across samples. The numbers of significant exons (FDR≤0.1) are indicated on the right.

FIG. 8B. Number of exons with lineage-specific splicing shifts (FDR≤0.1) identified in each BS-LR test.

FIG. 8C,D. Features associated with exons showing lineage-specific splicing shifts, including the percentage of exons with preserved reading frame (NMD_in and NMD_ex denote exons predicted to cause NMD upon exon inclusion or exclusion) (C) and with conserved splicing pattern in mouse (D). All cassette exons are used as control for comparison.

Simulation Analysis of p-Value Calibration and the Statistical Power of the EVE Model.

FIG. 9A. Validation of p-value calibration using data simulated based on the null hypothesis (no splicing shift between the two lineages in comparison.

FIG. 9B. Estimation of statistical power using data simulated with varying magnitudes of splicing shifts (average ΔΨ between 0.05 and 0.4) at p=0.05.

### Multiple Sequence Alignments of MAPT Exon 10.

Exonic splicing enhancers and silencers characterized in previous studies are indicated.

### MAPT Exon 10 Inclusion Across Different Human Brain Regions and Mouse Neuronal Cell Types.

FIG. 11A. Exon 10 inclusion across different brain regions as measured by RNA-seq data from BrainSpan. Brain regions are indicated at the top. Within each brain region, samples are ordered by developmental stages from embryonic (left) to adult (right) brains.

FIG. 11B. Exon 10 inclusion across mouse cortical neuronal and glial cell types as measured by Tasic et al. scRNA-seq data. For splicing quantification, RNA-seq reads were pooled for cells assigned to the same cell types. Glutamatergic and GABAergic neuronal cell types, as well as non-neuronal cell types, are indicated at the top.

### Expression of MBNL1/2 in HEK293 Cells as Compared to the Brain.

FIG. 12A. Immunoblots showing abundance of endogenous MBNL1 and MBNL2 in HEK293T cells and in the mouse brain at different developmental stages.

FIG. 12B. Immunoblots showing abundance of endogenous MBNL1 and exogenous MBNL2 without or with MBNL2 overexpression.

Efficiency of siRNA Knockdown of Endogenous MBNL1/2.

Top: Immunoblots showing abundance of endogenous MBNL1 and MBNL2 in HEK293T cells treated with siRNAs targeting MBNL1 (siMBNL1), MBNL2 (siMBNL2) or both (siMBNL1/2). siRNAs targeting GFP, scrambled siRNA sequences, and empty pcDNA plasmid, was used as controls. Bottom: Representative RT-PCR analysis of MAPT exon 10 inclusion in control and treated HEK293 cells.

### DETAILED DESCRIPTION OF THE EMBODIMENTS

One embodiment of the present technology presents a composition for treating a neurodegenerative disease, wherein the composition comprises at least one component that targets at least one muscleblind-like protein (MBNL) binding site near exon 10 of microtubule-associated protein tau (MAPT) to block MBNL binding thereto and modulate exon 10 splicing.

In another embodiment, the component targets at least two MBNL binding sites near exon 10 of MAPT, or specifically two MBNL binding sites near exon 10 of MAPT.

In another embodiment, the at least one component comprises nuclease-inactive dCas13d in complex with one or more guide RNAs (gRNAs). In yet another embodiment, the composition is useful for treating frontal temporal dementia (FTD).

In another embodiment, the at least one component comprises antisense oligonucleotide (ASO). In yet another embodiment, the composition is useful for treating frontal temporal dementia (FTD).

Another embodiment of the present technology provides a method of treating a neurodegenerative disease comprising the step of administering to a patient in need thereof an effective dose of a pharmaceutical composition which targets at least one muscleblind-like protein (MBNL) binding site near exon 10 of microtubule-associated protein tau (MAPT) to block MBNL binding thereto and modulate exon 10 splicing.

In another embodiment, the method of treatment is effective for treating frontal temporal dementia.

In yet another embodiment, the method of treatment comprises administering a composition that targets at least one two MBNL binding sites near exon 10 of MAPT, or specifically targets two MBNL binding sites near exon 10 of MAPT.

In a further embodiment, the method of treatment comprises administration of the pharmaceutical composition which composition comprises nuclease-inactive dCas13d in complex with one or more guide RNAs (gRNAs).

In a further embodiment, the method of treatment comprises administration of the pharmaceutical composition which composition comprises one or more antisense oligonucleotides (ASOs).

### EXAMPLES

### Example 1

### Systematic Identification of Lineage-Specific Splicing Shift Under Stabilizing Selection

To study quantitative changes in splicing during the evolution of the brain, we quantified exon inclusion of over 42,000 cassette exons in the brain using RNA-seq data of seven primate species with sequenced genomes, including two hominoids (human and Chimpanzee), three old world monkeys (rhesus macaque, crab-eating macaque and baboon) and two new world monkeys (marmoset and squirrel monkeys)^{23, 24} (Methods). At least two independent samples were available for each species, allowing assessment of intra-species variation, and exons with minimal splicing variation across species were excluded for downstream analysis. Unsupervised clustering readily identified groups of exons with differential splicing in specific lineages such as increased or decreased exon inclusion in humans and Chimpanzees (hominoids) compared to the other primate species (FIG. 7).

To identify divergent AS events with potential functional impact, we used an OU model to distinguish changes in selective constraints that result in a lineage-specific splicing shift in a phylogenetic tree against random drifts modeled as browning motion²⁵⁻²⁷ (FIG. 1A; see Methods). For each exon, five statistical tests were performed, including comparisons between human vs. non-human (both in all seven primate species or the five Catarrhine species), hominoids vs. non-hominoids (i.e., old-world and new-world monkeys), Catarrhine vs. new world monkeys. In total, we identified 1,170 exons showing significant shift in at least one test (FDR<0.1; FIG. 1B and FIG. 8A). Among them, we identified the largest number of exons with splicing shift in hominoids (n=725 compared to old world monkeys and n=541 compared to all other primates; FIG. 8B). Simulation analysis confirmed that the OUR model was well calibrated (FIG. 9A) and this method had a reasonable statistical power (e.g., over 80% power to detect a shift in exon inclusion level (DPSI)>0.25 between hominoids vs. old world monkeys or non-hominoid primate species at p=0.05; FIG. 9B).

About 50-60% of exons with lineage-specific shifts maintain the reading frame, which is comparable to cassette exons overall used for control (FIG. 8C). Exons displaying a more recent shift (e.g., human vs. all other primates) are less likely to have conserved AS patterns in mouse as an outgroup, but the proportion is nevertheless higher than all cassette exons, suggesting many of these exons have a relatively ancient origin (FIG. 8D). Intriguingly, we found that exons with human-specific shifts are more frequently under developmental stage-dependent regulation (FIG. 1C). Gene ontology (GO) analysis of genes harboring these exons suggest significant enrichment of genes involved in cilia assembly and morphogenesis in addition to terms reflecting a general feature of alternatively spliced gene (e.g., “protein binding”; FIG. 1D).

To further evaluate the reliability of the identified lineage-specific splicing shifts and gain mechanistic insights, we asked whether the splicing changes can be predicted from changes in splicing regulatory signals, as previous studies suggested that divergence in cis-acting splicing-regulatory elements is likely the major driving force of splicing divergence in mammals^{14, 34}. We expect mutations that create splicing enhancers and/or disrupt splicing silencers likely lead to an increase in exon inclusion, while mutations that disrupt splicing enhancers and/or create splicing silencers likely lead to a decrease in exon inclusion (FIG. 1E, upper panels). Indeed, when we examined the frequency of putative splicing-regulatory elements in the cassette exons and flanking intronic sequences, we found that a positive shift in exon inclusion is associated with a higher density of splicing enhancer elements and a decrease in density of silencer elements in the respective lineage (FIG. 1E, bottom panels). We also examined whether changes in the splice site strengths, predicted either using MaxEntScan (an entropy based method to score the consensus splice site motifs)³⁵ or SpliceAI (a deep learning method to predict splice site strength based on extended primary RNA sequences)³⁶ are consistent with changes in exon inclusion. In most cases, consistent changes were observed. MaxEntScan predicted ˜35% of exons showing consistent splicing changes vs ˜18% exons showing inconsistent exons, while ˜46% of exons do not have mutations in the splice site motif. Similarly, SpliceAI predicted ˜70% of exons with consistent splicing changes vs ˜30% with inconsistent splicing changes (FIG. 1F). The concordance between splicing changes and genomic sequence divergence lends support for the reliability of the exons we identified and also suggests that mutations affecting both the splice site motifs and splicing-regulatory elements outside the splice sites contribute to the lineage-specific splicing shift in primates.

### Example 2

### A Two-Step Lineage-Specific Shift in MAPT Exon 10 Splicing

We next focused on MAPT exon 10, which showed a significant shift in exon inclusion in hominoids as compared to old-world monkeys (Benjamini FDR=0.05) and all other primate species (Benjamini FDR=0.1) (FIG. 1B). The MAPT gene is located in chromosome 17q21, which is one of the most structurally complex and evolutionarily dynamic regions^{37, 38}. The encoded protein tau is frequently found in neurofilament tangles (NFT), a pathologic hallmark of multiple neurodegenerative diseases including Alzheimer's, FTD, Progressive supranuclear palsy (PSP), Pick's Disease, which are collectively referred to as tauopathy³⁹⁻⁴⁰. The AS of three developmentally regulated exons (i.e., exons 2, 3, and 10) result in the expression of six MAPT isoforms in the adult human brain (FIG. 2A). AS of exons 2 and 3 determine the number of N-terminal inserts (e.g., 0N, 1N, 2N), while exon 10 encodes the second of four microtubule binding repeats and its AS generates tau containing three (3R) or four (4R) repeats, which differ in microtubule binding affinity. Embryonic human brains express only 3R tau while adult human brains maintain a balance of 3R/4R tau at about an equal molar ratio. MAPT exon 10 splicing has been extensively studied in the literature, because mutations in or around this exon cause certain familial forms of FTD. Of particular importance, a subset of these mutations are synonymous or located in the flanking intronic regions, which do not affect protein sequences directly, but rather lead to an increase of exon 10 inclusion (FIG. 2B), suggesting that perturbation in the balance of 3R/4R tau isoform ratio is sufficient to cause the disease (see review¹⁸). Intriguingly, in mice, while exon 10 is initially skipped in the embryonic brains similar to humans, it increases during development to almost complete inclusion in the adult mouse brain, so that only 4R tau isoforms are expressed⁴¹. It is unclear when this divergence occurred during evolution.

The lineage splicing shift of MAPT exon 10 in primates detected by the OU model prompted us to perform a more systematic analysis to determine the evolutionary history of its splicing pattern divergence. Specifically, we examined exon inclusion in additional primate species with RNA-seq data available, including three hominoids (bonobo, gorilla, and orangutan), one old world monkey (baboon), and mouse lemur. Therefore, our extended analysis covered 12 sequenced primate species, including all but one (gibbon) sequenced hominoids. This analysis revealed that MAPT exon 10 splicing underwent a two-step lineage-specific splicing shift in primates during evolution. In new world monkeys and mouse lemur, the exon is almost completely included (>90%), while it reduces to 75-78% in old world monkeys, and ultimately to 41-54% in hominoids (FIG. 2C). Similar exon 10 inclusion level in chimpanzee, gorilla and gibbon as compared to human, as well as a higher inclusion in marmoset and, was previously noted in separate studies^{42, 43} and is consistent with our analysis.

Multiple splicing-regulatory elements in exon 10 were previously identified and some of these elements were disrupted by FTD-associated mutations leading to the increase of exon 10 inclusion (reviewed in re.⁴⁴). In addition, it was previously proposed that the sequence bordering exon 10 and intron 10 forms a stem loop structure, which works as a splicing repressor by blocking the accessibility of the 5′ splice site^{19, 20, 34, 45, 46} FTD-associated mutations overlapping with this region were predicted to destabilize this structure, leading to the increase of exon 10 inclusion in those patients. Several mutations were found in the orthologous sequence of mouse Mapt, which was also predicted to destabilize the structure and thus would explain the higher exon inclusion in the adult mouse brain⁴⁶⁻⁴⁸ (FIG. 2B). However, we found that the entirety of exon 10 and the predicted stem loop structure are completely conserved in all sequenced primate species (FIG. 2D and FIG. 10), disqualifying these splicing-regulatory elements from serving as the explanation of the observed splicing divergence.

### Example 3

### Divergence in MBNL-Dependent Regulation is a Major Driving Factor of MAPT Exon 10 Splicing Divergence

In considering the regulatory mechanisms driving the lineage-specific splicing shift of MAPT exon 10, we note the exon is almost completely skipped in embryonic brain in both human and mouse, despite the drastic difference in the adult brain. We thus hypothesized that divergence in splicing-regulatory elements important for developmental splicing switches may contribute to splicing pattern divergence.

This led us to focus on the Muscleblind splicing factor (MBNL) based on the following observations. We previously determined that MBNL1/2, whose expression increases during brain development, contribute to the developmental splicing switch of exon 10, as MBNL1/2 in the mouse brain led to a more “embryonic brain” like splicing pattern with reduction in exon 10 inclusion⁴⁹. Similarly, exon 10 inclusion was reduced in the postmortem brains of patients with myotonic dystrophy (DM), in which MBNL proteins are functionally sequestered due to an expansion of repetitive RNA containing MBNL binding sites⁵⁰.

To investigate MBNL-dependent splicing regulation, we performed RNA-seq to identify MBNL target exons by comparing wild type and MBNL1/2 double knockout (DKO) brain in mice and control and DM brains in human, and correlated the extent of MBNL-dependent splicing with developmental splicing switches. We previously demonstrated that MBNL is a master regulator of the second of two waves of developmental splicing switches (i.e., late splicing switches), which occurs in the first few months after birth in human and between P4 and P15 in mice during cortical development⁵¹.

Indeed, among exons with conserved developmental splicing switches at this time point between human and mouse, 65% and 42% are MBNL-dependent and the direction of MBNL-dependent splicing is consistent with that of developmental switches (FIG. 3A). Importantly, for exons with human-specific developmental changes and absence of switches in mice, there is significant reduction in MBNL-dependent splicing in mice (18% vs 65%, p=1.6e-6) but not in human (42% vs 42%, p=1), as compared to exons with conserved splicing switch patterns. For exons with mouse-specific developmental changes and absence of switches in human, there is significant reduction of MBNL-dependent splicing in human (7% vs 42%, p=5e-5), but not in mouse (60% vs 65%, p=0.55) (FIG. 3A). These data suggest MBNL-dependent regulation is a major driver of divergence in developmental splicing switches in mammals.

When we focused on MAPT exon 10, multiple lines of evidence suggest MBNL is a dominant regulator of its developmental splicing switch. First, exon 10 splicing switch occurs precisely at the time point when late switch occurs, and the human-mouse divergence is maximized in the adult brain. Second, we found the developmental splicing switch occur uniformly across different cortical regions (FIG. 11A), consistent with the wide expression pattern of MBNL. Third, exon 10 is consistently included at a high level (˜95%) across diverse neuronal and glial cell types in the adult mouse cortex (FIG. 11B), consistent with the expression and activity of MBNL not only in neurons but also in glia. In line with this notion, a developmental splicing switch of exon 10 was also observed in rodent oligodendrocytes⁵².

Importantly, the magnitude of MBNL-dependent activation is greater in mice than in human (ΔY=0.23 in human and 0.56 in mouse; FIG. 3B,C), which led us to hypothesize that attenuation of MBNL-dependent regulation may explain the lineage-specific splicing shifts in hominoids and old-world monkeys as compared to more distal primates and rodents. We noted that the exon skipping is not complete in MBNL1/2 DKO mouse brains or DM human brains, which is likely due to incomplete depletion or functional sequestration of MBNL. To test our hypothesis, we first examined the range of exon inclusion that can be driven by different levels of MBNL expression.

To this end, we generated human and mouse MAPT (hMAPT and mMAPT) minigenes encompassing MAPT exon 9 to exon 11, which was transfected into HEK293T cells that have a relatively high expression level of MBNL1 but a low level of MBNL2, as compared to the brain (FIG. 12A, B). In this condition, exon 10 is included at 11-25% for hMAPT and 24-54% for mMAPT (FIG. 3D and FIG. 4 below). Importantly, knockdown of MBNL1/2 by co-transfection of specific siRNAs led to nearly complete skipping of both hMAPT and mMAPT exon 10 (<2%), while overexpression of a FLAG tagged MBNL2 (FLAG-MBNL2) increases the exon inclusion level to 40-58% for hMAPT and 60-75% for mMAPT (FIG. 3D, FIG. 4 and FIG. 13). These data suggest MBNL-dependent regulation largely explains the range of exon inclusion levels observed during brain development and that the extent regulation is attenuated in human as compared to mouse.

### Example 4

### Divergence in MBNL Binding Sites Contribute to MAPT Exon 10 Splicing Divergence

MBNL binds clusters of YGCY elements to regulate AS⁵³⁻⁵⁵. We next searched for splicing-regulatory elements required for MBNL-dependent inclusion of MAPT exon 10 and evidence whether divergence of these elements can explain the splicing divergence observed both across primate species and between primates and mice. Taking advantage of the MBNL2 CLIP data we previously generated from mouse and human brains^{49, 50} and bioinformatically predicted MBNL-binding motif sites (YGCY clusters)⁵³, we found two major MBNL binding sites (denoted site 1 and site 2) in intron 10 that are supported by both CLIP data and motif-based predictions (FIG. 4A). Site 1 is located deep in the middle of intron 10 (chr17:44,089,181-44,089,432, hg19), and appears to have a higher binding affinity in human, while site 2 is ˜550 bp upstream of exon 11 (chr17:44,090,854-44,090,942, hg19), and appears to be stronger in mice. There is an extensive turnover of YGCY elements that occurred in different lineages that may lead to differences in MBNL binding affinity (e.g., 4 YGCYs in humans and 7 YGCYs in mice for site 2; FIG. 4A). These data suggest a possibility that divergence in MBNL binding sites in the downstream intron can change the magnitude of MBNL-dependent splicing activation and thus explain the lineage-specific splicing shifts in the adult brains.

To validate the importance of the identified MBNL binding sites in the regulation of exon 10 splicing, and its contribution to lineage-specific shifts, we performed a series of mutagenesis in the predicted MBNL binding sites using hMAPT and mMAPT minigenes. We first generated truncation mutants of the mMAPT minigene by eliminating MBNL binding site 1, site 2, or both sites (FIG. 4B). The wild type or mutant minigene reporters were transfected into HEK293T cells, with or without co-transfection of FLAG-MBNL2. An individual deletion of site 1 or site 2 reduced exon 10 inclusion from 42% to about 15%, and simultaneous deletion of both sites resulted in even more of a reduction to <9% (FIG. 4B). Overexpression of MBNL2 increased exon 10 inclusion in all of the minigene constructs, especially for the wild type and single site deletion mutants, data that is consistent with MBNL-dependent exon activation (FIG. 4B). Deletion of either site 1 or site 2 resulted in a partial reduction in exon 10 inclusion, and the largest reduction was again observed when both binding sites were deleted. These data suggest that both site 1 and site 2 are important for MBNL-dependent splicing regulation of exon 10. Moreover, the MBNL-dependent increase in exon 10 inclusion is diminished for the double deletion mutants, suggesting that these two sites represent the most critical regulatory elements for MBNL-dependent splicing regulation.

We hypothesized that site 2 might be more relevant for the lineage-specific splicing shifts of exon 10 across species, given the predicted weaker binding of MBNL in human than in mice, a pattern that is consistent with the divergent exon inclusion levels. To test this hypothesis, we replaced the mouse site 2 sequence with the orthologous human sequence (FIG. 4A). Interestingly, exon 10 inclusion levels in the minigene that carries human site 2 sequence did not rescue the reduction of exon inclusion caused by site 2 deletion, confirming that the MBNL2 binding site 2 has reduced activity in human as compared to the mouse counterpart (FIG. 4C).

We next performed additional MBNL binding site replacement experiments using hMAPT minigene. Human site 2 sequence replaced with the mouse sequence led to an increase in exon 10 inclusion in both conditions without or with overexpression of MBNL2, again consistent with our hypothesis that mouse site 2 has a higher binding affinity to MBNL than the human site 2. Interestingly, the effect of site 1 replacement appears to depend on the expression level of MBNL. Without overexpression of MBNL2, human site 1 replaced with the mouse sequence reduced exon 10 inclusion. However, upon MBNL2 overexpression, which is more similar to the brain with respect to the MBNL expression level, site 1 replacement led to a slight increase, if any, in exon 10 inclusion (FIG. 4D). Together, these experiments confirmed that MBNL binding sites, especially site 2, are more potent in activating exon 10 inclusion in the mouse gene than in the human gene.

We next replaced the site 2 sequence in the hMAPT minigene to its orthologous sequences from rhesus macaque as representative of old-world monkeys, and marmoset and squirrel monkey as representatives of new world monkeys. Replacement of human site 2 to marmoset and squirrel monkey sequences increased exon 10 inclusion compared to the wild type hMAPT minigene without or with MBNL2 overexpression, again consistent with our hypothesis. Replacement with rhesus monkey sequence does not have significant impact (FIG. 4E). Overall, these data support our hypothesis that attenuation of MBNL-dependent splicing regulation caused by weakening of MBNL binding sites is a major factor contributing to lineage-specific splicing shift of MAPT exon10 in hominoids and the old world monkeys.

### Example 5

Modulation of MAPT Exon 10 Splicing by Steric Hindrance Using dCas13d/gRNAs

Given the clinical relevance of MAPT exon 10 inclusion in FTD and potentially other tauopathies, previous studies attempted to modulate exon 10 splicing by targeting splice sites and/or exonic splicing regulatory elements using antisense oligos (ASOs)⁵⁶ or the RNA-targeting CRISPR system composed of the enzymatically dead Cas13d (dCas13d) in complex with guide RNAs (gRNAs)⁵⁷. We thus tested whether the MBNL binding sites we identified can be used to modulate splicing with potential applications in the clinical settings. Specifically, we used dCas13d/gRNA to target the site 2 sequence in the mMAPT minigene and antagonize recognition of the site by MBNL proteins and thus MBNL-mediated splicing. Four gRNAs were designed to target the MBNL site 2 sequence containing YGCY elements. These gRNAs, individually or all together, were co-transfected along with the mMAPT minigene in HEK293T cells (FIG. 5A). We found expression of dCas13/gRNAs reduced exon 10 inclusion, with the strongest inhibition observed when gRNA3 or all four gRNAs together were expressed (FIG. 5B). When the experiment was performed with simultaneous overexpression of MBNL2, individual gRNAs in general were not sufficient to compete against MBNL, except for gRNA3, but simultaneous expression of all four gRNAs again blocked the MBNL-RNA interaction and exon 10 inclusion.

### Example 6

### Modulation of MAPT Exon 10 Splicing by Steric Hindrance Using ASOs

We next tested whether ASOs can be used to target the MBNL binding sites and antagonize MBNL binding, thereby modulating splicing of exon 10. Specifically, we designed 15 ASOs that overlap with predicted MBNL binding YGCY elements and performed a screen (FIG. 6A and Table 1). These ASOs have 2′-O-methoxyethyl (MOE) with a phosphorothioate backbone (2′MOE-PS) for each nucleotide and the chemistry has been successfully used in treatment of spinal muscular atrophy (SMA) and a number of clinical studies. Each ASO was individually transfected into HEK293T cells together with the hMAPT minigene, and 48 hours after transfection, cells were collected to extract RNA and perform RT-PCR analysis. We found several ASOs demonstrated robust effects in reducing exon 10 inclusion, with ASO 1 and ASO 4 showing the strongest effect (FIG. 6B), suggesting that ASOs targeting MBNL binding sites can potentially be used to modulate exon 10 splicing in the clinical settings.

TABLE 1
antisense oligo nucleotides (ASOs) tested for MAPT exon 10 splicing modulation.

| ASO ID | ASO name | ASO seq | Target seq |
|---|---|---|---|
| ASO1 | MAPT_10_20_1 | GGGCAGGAAGCAGCAGCACA | TGTGCTGCTGCTTCCTGCCC |
| | | (SEQ ID NO: 1) | (SEQ ID NO: 2) |
| ASO2 | MAPT_10_20_2 | AGCAGCCGCAGGGCAAGCGC | GCGCTTGCCCTGCGGCTGCT |
| | | (SEQ ID NO: 3) | (SEQ ID NO: 4) |
| ASO3 | MAPT_10_20_3 | AGCGTTAGTTAGCCCCATGC | GCATGGGGCTAACTAACGCT |
| | | (SEQ ID NO: 5) | (SEQ ID NO: 6) |
| ASO4 | MAPT_10_20_4 | AGCAGACACTGGTGAGGAAG | CTTCCTCACCAGTGTCTGCT |
| | | (SEQ ID NO: 7) | (SEQ ID NO: 8) |
| ASO5 | MAPT_10_20_5 | CAGGAGAGCAGGGCGTGGGA | TCCCACGCCCTGCTCTCCTG |
| | | (SEQ ID NO: 9) | (SEQ ID NO: 10) |
| ASO6 | MAPT_10_20_6 | GGCGGCACACACCTCTTCTG | CAGAAGAGGTGTGTGCCGCC |
| | | (SEQ ID NO: 11) | (SEQ ID NO: 12) |
| ASO7 | MAPT_10_20_7 | GGGTGGGGGCGGCACACACC | GGTGTGTGCCGCCCCCACCC |
| | | (SEQ ID NO: 13) | (SEQ ID NO: 14) |
| ASO8 | MAPT_10_20_8 | GGCAGGGGTGGGGGCGGCAC | GTGCCGCCCCCACCCCTGCC |
| | | (SEQ ID NO: 15) | (SEQ ID NO: 16) |
| ASO9 | MAPT_10_20_9 | GTAGGTGGCACAGCAGAAAC | GTTTCTGCTGTGCCACCTAC |
| | | (SEQ ID NO: 17) | (SEQ ID NO: 18) |
| ASO10 | MAPT_10_20_10 | TAGCAATAGGAACAAAGCAA | TTGCTTTGTTCCTATTGCTA |
| | | (SEQ ID NO: 19) | (SEQ ID NO: 20) |
| ASO11 | MAPT_10_20_11 | AGAACCAGCAGGCACCTGCA | TGCAGGTGCCTGCTGGTTCT |
| | | (SEQ ID NO: 21) | (SEQ ID NO: 22) |
| ASO12 | MAPT_10_20_12 | GACCCAGCGGAGTTCAGCAG | CTGCTGAACTCCGCTGGGTC |
| | | (SEQ ID NO: 23) | (SEQ ID NO: 24) |
| ASO13 | MAPT_10_20_13 | TAAGCAGGACCCAGCGGAGT | ACTCCGCTGGGTCCTGCTTA |
| | | (SEQ ID NO: 25) | (SEQ ID NO: 26) |
| ASO14 | MAPT_10_20_14 | GCAAAGACCATCAGTAAGCA | TGCTTACTGATGGTCTTTGC |
| | | (SEQ ID NO: 27) | (SEQ ID NO: 28) |
| ASO15 | MAPT_10_20_15 | GGAAAGCACTAGAGCAAAGA | TCTTTGCTCTAGTGCTTTCC |
| | | (SEQ ID NO: 29) | (SEQ ID NO: 30) |

### Prophetic Example 7

These methods will also be useful for expressing and identifying MAPT splice isoformns found in the adult brain, which can be used for targeted modeling of tauopathies. Examples 3 and 4 show that overexpression of MBNL1 or MBNL2 increased exon 10 inclusion, so overexpression of MBNL proteins can switch MAPT splicing from embryonic isoforms to adult isoforms. Thus, the described methods involve manipulating MBNL expression level in cells (for example HEK293T cells) to promote generation of tau isoforms that are expressed in adult brains and implicated in tauopathies. As noted above, various ASOs can be used to target MBNL binding sites, resulting in alteration of different MAPT isoforms and correction of splicing patterns, providing an in vitro system to study disease mechanisms and evaluate therapeutic interventions for disease treatment.

### DISCUSSION

We investigated divergent AS in primate brains and demonstrated that 2.7% of annotated cassette exons (from ˜900 genes) show evidence of lineage-specific splicing shifts under stabilizing selection. The largest number of these exons have shifted splicing in hominoids. These exons, especially those showing human-specific splicing shifts, tend to be regulated during brain development, suggesting their potential functional relevance. Mechanistically, the splicing shifts can be explained by mutations in both splice sites as well as numerous splicing-regulatory elements outside the splice sites, although precise prediction of mutations leading to divergent splicing of individual exons is challenging at the current stage. Among the exons we identified, of particular interest is AS of MAPT exon 10, given its drastic developmental regulation, the relevance of this exon in FTD, and the previously noted divergence of exon inclusion level in human and mouse brains. MAPT tau function has been studied extensively in the context of neurodegeneration due to NFT formed by insoluble tau as a hallmark of the pathology. Exon 10 splicing is believed to play an instrumental role as different forms of NFT consisting of distinct 3R/4R isoform compositions underly different types of neurodegenerative diseases and perturbation of 3R/4R is sufficient to cause FTD^{39, 40}.

The physiological function of MAPT, and the impact of evolutionary changes in the gene and the 17q21 genomic locus, is more controversial. Most of these functional studies were performed using rodent models, and the interpretation of the results might have been complicated by additional evolutionary differences in other parts of the gene, as compared to human (e.g., ref.⁵⁸). In human, 17q21.3 microdeletion encompassing the MAPT gene can cause severe developmental delays and intellectual disability^{59, 60}. The susceptibility to microdeletion and neurodegeneration is proposed to be associated with two haplotypes of this locus^{60, 61}. The divergence of exon 10 splicing between human and mouse, despite a high level of conservation in the exon 10 sequence, has been a long-standing observation, and raised an intriguing question whether or how different species have different susceptibility to neurodegeneration^{62, 63}.

This study revealed remarkable evolutionary shifts of exon 10 splicing under stabilizing selection in different lineages of primate species, and how mutations disrupting distal splicing regulatory elements resulted in the splicing shift at specific developmental stages. Our results suggest an adaptation of MAPT function in primates during neurodevelopment and may also have implications in neurodegeneration. This example may represent a more general mechanism how divergence in splicing could contribute to speciation.

### Methods

### Primate Brain RNA-Seq Data Analysis.

To model splicing shift of cassette exons in specific lineage in a phylogenetic tree, we used adult brain RNA-seq data from human²³ and six other primate species (data from non-human primate reference transcriptome resource, NHPRTR²⁴). To evaluate MAPT exon 10 splicing evolution, we used additional RNA-seq data from adult brain of primate species to cover more species and increase the number of biological replicates from the same species⁶⁴. Only species with sequenced reference genome and at least two biological replicates in each RNA-seq dataset were included in our analysis.

To evaluate divergence of exon inclusion level in primates, we mapped RNA-seq raw reads to the human reference genome (hg19) allowing 8 mismatches (for 101 nt reads). The number of allowed mismatches were relaxed here compared to standard analysis to accommodate mismatches caused by evolutionary changes in genomic sequences. This approach was found to be preferred over mapping to the individual reference genome for each species due to varying quality/completeness across the reference genomes and the complication of mapping errors due to pseudogenes that affect each species differently.

Quantification of splicing for 42,761 previously annotated cassette exons was performed using the Quantas pipeline (https://zhanglab.c2b2.columbia.edu/index.php/Quantas), as we described previously⁶⁵. In brief, the inclusion level of each cassette exon (percent spliced in Y) was calculated from the number of supporting exon junction reads for the inclusion and skipping isoforms, and only quantifications with ≥20 supporting junction reads were used for downstream analysis (and missing value was assigned otherwise).

### Using an OU Process to Model AS Divergence as a Quantitative Trait.

For preprocessing, we first excluded exons unless they can be quantified in at least one sample for each of the seven primate species. For the remaining exons, missing values were imputed using Bayesian PCA method in the R package ‘pcaMethods’⁶⁶. Then, exons with minimal variation across samples (as measured by standard deviation (SD)<0.05) were excluded. In total, 3,690 cassette exons passed these filtering steps and were used for OU modeling (Supplementary Table 1).

Compared to other methods, such as generalized linear models (GLMs), the OU model provides a statistical framework to model evolutionary changes of a quantitative traits in large phylogenies with more complex covariance structure to distinguish stabilizing selection and random drift. This method has been previously adapted to analyze evolution of gene expression⁶⁴, but not splicing. In brief, the OU process used to model splicing of each exon across species in a phylogeny can be viewed as a random walk (Brownian motion) plus a pull towards an optimal value^{26, 27}.

More formally, dΨₜ=α(θ−Ψₜ)dt+σdWₜ, where Ψₜ is the quantitative trait value (i.e., exon inclusion level in our case) at time t, α parameterizes the strength of pull towards the optimal value θ. dWₜ models evolutionary drift using a normally distributed random variable with variance dt, and σ parameterizes the strength of drift. In this framework, the change of the quantitative trait over a time interval dt is the sum of a stochastic component (σdWₜ, drift) and a deterministic component (α(θ−Ψₜ), stabilizing selection). For a phylogeny with known topology and branch lengths, we define r selective regimes acting on the phylogeny, each regime defined by an optimal value θ∈(θ₁, . . . , θᵣ). We can then assign an OU process with parameters α, σ², and θᵢ to model the trait value of each taxa Ψᵢ. Now let Ψᵢ=[Ψ₁, Ψ₂, . . . , Ψ_{N}] be the state of the OU processes at the N terminal taxa; Ψ follows a multivariate normal distribution. These model parameters can be estimated the standard maximum likelihood methods.

Using the framework, we can identify AS exons under differential stabilizing selection in particular lineages (i.e., two selective regimes in the simplest case as described in this study) by testing the null hypothesis (in which all branches share the same optimum parameters θ_{shared}) against the alternative hypothesis (in which a different optimum θ₁≠θ_{shared} acted on a particular lineage). A likelihood ratio (LR) test can be used to assess the fitness of the null and alternative models using a chi-square distribution, since alternative hypothesis has one additional degree of freedom as compared to the null hypothesis (LRT_{βi≠βshared}˜χ₁²), which describes the ratio of population to evolutionary expression/splicing level variance. Here, βᵢ is for individual gene i, reflecting the evolutionary process of gene i based on its degree of expression diffusion and constraint. β_{shared} is the shared β of all genes controlling the population expression/splicing variance.

We applied a phylogenic ANOVA test, as implemented in the Expression Variance and Evolution (EVE) model²⁵ with a phylogenetic tree downloaded from the UCSC genome browser (https://genome.ucsc.edu). We applied this method to run five branch-specific (BS)-LRT tests on different selective regimes (i.e., human vs. non-human, hominoid vs. non-hominoid, Catarrhini vs new world monkey, either in the whole phylogenetic tree composed of seven species or in Catarrhini composed of five species), as summarized in Supplementary Table 1. For tests performed for each lineage, chi-square p-values were adjusted by Benjamini & Hochberg multiple test correction to obtain false discovery rate (FDR).

### Power Estimation for EVE Model in Detecting AS Exons Under Adaptive Evolution.

We performed a series of simulations to evaluate the p-values and estimate the power of the EVE method in detecting adaptive splicing divergence between different branches in the phylogenetic tree. For each BS-LR test, we simulated the inclusion levels of 100 exons under a null model of θ_{shared} across the phylogeny, using parameters estimated from the same BS-LR test on real RNA-seq data. All simulated Y values were limited between 0 and 1 (Y values <0 were set to 0 while Y values >1 were set to 1). The simulated data was then subjected to BS-LR test, and the p-value for each exon was calculated using chi-square test with df=1. The observed and expected p-values were compared using a qq-plot (FIG. 8) to confirm whether the chi-square p-values were properly calibrated. To estimate the power, we simulated 100 exons with a branch-specific shift in exon inclusion (Δθ) in one lineage relative to the other branch in the phylogenetic tree. We calculated the power of the EVE model in detecting adaptive evolution by counting how many times the simulated exons were called significant (chisq p-value<0.05). These simulations were performed for varying magnitudes of branch-specific shifts, (Δθ ranging from 0.01 to 0.4), reflecting weak or drastic branch-specific adaptive selection.

### Annotation of Cassette Exons.

To annotate cassette exons with lineage-specific splicing shifts, we obtained conservation of AS in mice and the impact of AS on protein-coding from a previous study⁸. Developmental-splicing changes were evaluated using human brain RNA-seq data from ref.²³ (Lister) and BrainSpan⁶⁷. Briefly, pairwise differential splicing analysis were performed using samples from different age groups for the Lister and BrainSpan datasets, separately (coverage≥20, |Δψ|≥0.2, and Benjamini FDR≤0.05). We excluded exons with significant changes only in one comparison for human to focus on more robust changes and avoid potential confounding factors (such as variation in sample quality or variation in genetic backgrounds among individuals). In total, 4651 cassette exons are called to have developmental splicing switches. Gene ontology (GO) analysis were performed for each list of genes containing cassette exons with lineage-specific splicing shift and GO terms that are significant in at least one gene list (Benjamini FDR<0.05) were shown in FIG. 1D.

### Analysis of Splicing-Regulatory Elements Among Divergent Cassette Exons.

Exonic and flanking intronic sequences of all human cassette exons were extracted from the human reference genome (hg19). We also mapped the coordinates of cassette exons from hg19 to the other reference genomes using UCSC's liftOver tool to retrieve the homologous sequences in the other non-human primate species (the assembly version of other non-human primate species can be found in Supplementary Table 1).

For exons showing lineage-specific splicing switches, we sought to test whether increase in exon inclusion is associated with increase of splicing enhancers and/or decrease in splicing silencers, while decrease in exon inclusion is associated with decrease of splicing enhancers and/or increase in splicing silencers. To this end, we analyzed lists of computationally identified splicing-regulatory elements from previous studies, including RESCUE ESE⁶⁸, FAS-ESS⁶⁹, Chasin-PESE and PESS⁷⁰, exon identity element (EIE) and intron identity elements (IIE)⁷¹. For each group of exons showing a specific divergence pattern (e.g., higher inclusion in hominoids compared to other primate species), we calculated the density of each type of features in the exon or upstream/downstream flanking intronic regions (100 nt) using sequences from the two compared lineages (observed counts normalized by the total possible counts determined by total sequence lengths). The density was compared between the two lineages using a two-sided Binomial test.

We also considered whether the splicing shift can be explained by changes in splice site strength. For this analysis, splice site strength was calculated using MaxEntScan, which scores the splice site motifs³⁵ and SpliceAI, a machine learning-based algorithm that predict splice site using both local and distal sequences³⁶. For MaxEntScan, the prediction scores of 3′ss and 5′ss were averaged as a measure of splice site strength of the exon. For SpliceAI, we used the maximum of the 5′ss and 3′ss score as a measure of splice site strength of the exon³⁶. The numbers of exon pairs with consistent and inconsistent directions are summarized in FIG. 1F.

### Comparative Analysis of Developmental Splicing Regulation in Human and Mouse Cortex.

We compared the developmental splicing profiles of 3583 pairs of orthologous exons in human and mouse developing brains using two datasets of human brain development^{23, 67} and two datasets of mouse brain development^{23, 65}. Exons with developmental splicing switches at specific time points were identified using WGCNA analysis in combination with sigmoidal fits, as described previously⁵¹. More detailed procedure will be described else well. In this study, we focused on exons showing late splicing switches and increased exon inclusion during brain development, including 81 exons with conserved switches, 42 exons with mouse-specific switches and 38 with human-specific switches.

### RNA-Seq Analysis of Mbn1-Dependent Splicing in Human and Mouse Cortex.

To identify the comprehensive list of Mbn1-dependent exons, we previously generated a Mbnl1^{-/-}; Mbn12^{loxP/loxP}; Nestin-Cre line to deplete both Mbnl1 and Mbnl2 in neurons and glial cells (referred to Mbn1 double-KO or DKO). Deep RNA-Seq was performed using RNA extracted from adult Mbn1 DKO and control cortices using the standard Illumina TruSeq platform (PE 101-nt reads) (SRA accession: SRP142522). In parallel, we also performed RNA-Seq analysis of human cortex obtained from control and myotogenic dystrophy type 1 (DM1) patients, each group in triplicates (data are being deposited to SRA). Raw RNA-Seq reads were processed using the same Quantas pipeline, as described above. Differential splicing was called in mouse and human with the following criteria (coverage≥20), |ΔI|≥0.1, and Benjamini FDR≤0.05).

### Plasmid Cloning of MAPT Minigenes.

The human MAPT (hMAPT) mouse Mapt (mMAPT) minigenes containing exon 9, exon 10, and exon 11 with relevant intronic sequences were cloned using Gibson assembly into the backbone vector pcDNA5/FRT (ThermoFisher Scientific, Waltham, MA) using the multiple cloning site (MCS) HindIII and XhoI restriction sites (see primer and gblock sequences listed in Supplementary Table 2). For mMAPT minigene, the genomic region of interest was PCR amplified from purified DNA obtained from wild type B6/C57 mouse tail. Part of intron 9 (chr11: 104,310,938-104,317,156) is truncated to limit the size of the minigene. In addition, a total of four mutant mMAPT minigenes with MBNL2 binding site deletions were cloned with Gibson assembly: I) deleted mouse MBNL2 binding site 1, II) deleted mouse MBNL2 binding site 2, III) both mouse MBNL2 binding site 1 and 2 deleted, and IV) mouse MBNL2 binding site 2 replaced with the human MBNL2 binding site 2 (chr17: 44,090,858-44,090,933). For hMAPT minigene, genomic region of interest was PCR amplified using DNA purified from HEK293T cells, and part of intron 9 (chr17:44,074,434-44,086,750) orthologous to the truncated mouse sequence was also truncated. In addition to the wild type hMAPT minigene, we also generated four mutant hMAPT minigenes in which the MBNL2 binding site 2 was replaced with the binding site 2 sequence from I) rhesus macaque, II) squirrel monkey, III) marmoset monkey, and IV) mouse. All primer and gblock sequences used for cloning are listed in Supplementary Table 2. All plasmids generated were confirmed by Sanger sequencing (Eton Bioscience Inc., Union, NJ).

### MAPT Minigene Splicing Reporter Assay.

For human and mouse MAPT splicing reporter assays, HEK293T cells maintained in Dulbecco's modified Eagle's medium (DMEM) and supplemented with 10% FBS were seeded one day before transfection (about 3.5×10⁵ cells per well of a 6-well plate). Plasmid DNA (0.125 μg minigene+1.5 μg pCAGGS-3×FLAG-MBNL2 expression vector, 1.625 g total) was transfected using Lipofectamine 3000 (Thermo Fisher Scientific, Waltham, MA) according to manufacturer's instructions. Twenty-four hours post transfection cells were scrapped in ice-cold 1×PBS and spun down. Half of the cells were used for total RNA isolation using Trizol reagent (Thermo Fisher Scientific, Waltham, MA) and Direct-zol RNA kits (Zymo Research). The remaining cells were resuspended in 60 μl lysis buffer (50 mM HEPES, pH 7.4, 100 mM NaCl, 1% Triton X-100, 0.1% sodium dodecyl sulfate (SDS), 1 mM EDTA, 1 mM dithiothreitol (DTT), cOmplete™ protease inhibitors (Roche)) for protein extraction. Protein lysates from mouse cortices at different ages (P4, P7, P30) were generated in a previous study⁸.

To confirm protein expression using immunoblot, protein samples were prepared with 2× Laemmlin Sample Buffer (Bio-Rad Laboratories, Hercules, CA) and 89 mM 0-mercaptoethanol, boiled, and loaded into 4-12% SDS-polyacrylamide gel electrophoresis (SDS-PAGE) Novex Bis-Tris gels (Thermo Fisher Scientific, Waltham, MA). After protein transfer onto 0.45 m nitrocellulose membrane (GE Healthcare, Marlborough, MA), the following primary antibodies were used: rabbit α-MBNL1 (A2746, generous gift from Charles Thornton, 1:1000), mouse α-MBNL2 (3B4, SCBT), mouse α-FLAG M2 (Sigma-Aldrich, F1804, 1:4000), and mouse α-GAPDH (EMD Millipore, CB1001, 1:10,000).

For RT-PCR analysis of the splicing products, cDNA was prepared using SuperScript III reverse transcriptase (Thermo Fisher Scientific, Waltham, MA) with random hexamer primers. To measure exon inclusion, alternative exons of interest were amplified with primers listed in Supplementary Dataset. PCR products were resolved on 1.2-1.5% agarose gel.

### MBNL1/2 RNA Interference and MBNL2 Overexpression.

For samples with knockdown of MBNL1 and MBNL2, siRNAs (Millipore Sigma; MBNL1: 100 nM; MBNL2: 100 nM) were transfected into HEK293T cells seeded the day before (about 3.0×10⁵ cells per well of a 6-well plate) using Lipofectamine 3000 (ref.⁷²). The MAPT minigene plasmids (125 ng) were transfected 24 hr after siRNA transfection using Lipofectamine 3000, as well. For overexpression of MBNL2, (1.5 ug) pCAGGS-3×FLAG-MBNL2 expression vector was transfected at the same time point with MAPT minigene plasmids⁷³. RNA and protein were collected 48-hour post minigene transfection for immunoblots and RT-PCR analysis of the splicing products, as described above.

Blocking of MBNL Binding Site 2 Using dCas13dgRNA.

A total of four individual gRNAs targeting MBNL binding site #2 in the mouse Mapt (mMAPT) minigene splicing reporter were cloned. The oligonucleotides were synthesized (IDT) and cloned in the CasRx gRNA cloning backbone using BsmBI restriction site (Addgene plasmid #138150). gRNA targeting sequences can be found in Supplementary Table 2. All constructs were confirmed by Sanger sequencing (Eton Bioscience Inc., Union, NJ).

For gRNA/dCas13d transfections, HEK293T cells maintained in DMEM and supplemented with 10% FBS were seeded one day before transfection (about 3.5×10⁵ cells per well of a 6-well plate). A plasmid ratio of mMAPT:gRNA:dCas13d (1:32:32 ng) was transfected using Lipofectamine 3000 (ThermoFisher) according to manufacturer's instructions (dCas13d: Addgene plasmid #109050). In samples to test the effect of MBNL2 overexpression, 625 ng of pCAGGS-3×FLAG-MBNL2 expression vector was co-transfected, along with the mMAPT minigene and dCas13d/gRNA plasmids. Cells were collected 48-hour post-transfection for RNA and protein extraction. Immunoblots and RT-PCR analysis of splicing products were performed as described above.

### Blocking of MBNL Binding Site Using ASOs.

A total of 15 ASOs targeting MBNL binding sites in the human Mapt (hMAPT) minigene splicing reporter were screened to modulate exon 10 splicing (Table 1 for ASO sequences). ASOs with 2′MOE-PS chemistry were obtained from integrated DNA technologies (IDT). HEK293T cells were maintained as described above. ASOs were transfected at 80 nM concentration together with the 25 ng hMAPT minigene plasmid using Lipofectamine 3000 (ThermoFisher), according to manufacturer's instructions. Cells were collected 48-hour post-transfection for RNA extraction. RT-PCR analysis of splicing products were performed as described above.

### A Method to Model Tauopathies Using In Vitro Systems

Given the inaccessibility of brain tissues from tauopathy patients, especially during the early stages of the diseases, and the evolutionary difference of MAPT splicing between human and other model organisms, such as rodents, there is tremendous interest to model tauopathies using in vitro systems, such as neurons or brain organoids derived from induced pluripotent stem cells^{75, 76}. One critical challenge for these efforts is to express the correct tau isoforms implicated in pathologies. For example, neurons differentiated in vitro from stem cells are in general immature, and it was reported that even after extended culture (e.g., 1 year), these cells still predominantly express the embryonic isoforms^{77, 78}. Results disclosed in this invention reveal MBNL proteins as instrumental regulators of the developmental regulation of tau splicing isoforms during neuronal development. We note that these results suggest a method to manipulate MBNL expression level in cells to promote generation of tau isoforms that are expressed in adult brains and implicated in tauopathies.

### REFERENCES

1. Berget, S. M., Moore, C. & Sharp, P. A. Spliced segments at the 5′ terminus of adenovirus 2 late mRNA. Proc. Natl. Acad. Sci. USA 74, 3171-3175 (1977).
2. Chow, L. T., Gelinas, R. E., Broker, T. R. & Roberts, R. J. An amazing sequence arrangement at the 5′ ends of adenovirus 2 messenger RNA. Cell 12, 1-8 (1977).
3. Gilbert, W. Why genes in pieces?Nature 271, 501-501 (1978).
4. Sousa, A. M. M., Meyer, K. A., Santpere, G., Gulden, F. O. & Sestan, N. Evolution of the human nervous system function, structure, and development. Cell 170, 226-247 (2017).
5. Wang, E. T. et al. Alternative isoform regulation in human tissue transcriptomes. Nature 456, 470-476 (2008).
6. Pan, Q., Shai, O., Lee, L. J., Frey, B. J. & Blencowe, B. J. Deep surveying of alternative splicing complexity in the human transcriptome by high-throughput sequencing. Nature Genet 40, 1413-1415 (2008).
7. Castle, J. C. et al. Expression of 24,426 human alternative splicing events and predicted cis regulation in 48 tissues and cell lines. Nat Genet 40, 1416-1425 (2008).
8. Yan, Q. et al. Systematic discovery of regulated and conserved alternative exons in the mammalian brain reveals NMD modulating chromatin regulators. Proc Natl Acad Sci USA 112, 3445-3350 (2015).
9. Zhang, Y. et al. An RNA-sequencing transcriptome and splicing database of glia, neurons, and vascular cells of the cerebral cortex. J Neurosci 34, 11929-11947 (2014).
10. Weyn-Vanhentenryck, S. et al. HITS-CLIP and integrative modeling define the Rbfox splicing-regulatory network linked to brain development and autism. Cell Rep. 6, 1139-1152 (2014).
11. Modrek, B. & Lee, C. J. Alternative splicing in the human, mouse and rat genomes is associated with an increased frequency of exon creation and/or loss. Nat. Genet. 34, 177-180 (2003).
12. Lev-Maor, G. et al. Intronic Alus influence alternative splicing. PLoS Genetics 4, e1000204 (2008).
13. Merkin, J., Russell, C., Chen, P. & Burge, C. B. Evolutionary dynamics of gene and isoform regulation in mammalian tissues. Science 338, 1593-1599 (2012).
14. Barbosa-Morais, N. L. et al. The evolutionary landscape of alternative splicing in vertebrate species. Science 338, 1587-1593 (2012).
15. Reyes, A. et al. Drift and conservation of differential exon usage across tissues in primate species. Proc Natl Acad Sci USA 110, 15377-15382 (2013).
16. Gao, Q. S. et al. Complex regulation of tau exon 10, whose missplicing causes frontotemporal dementia. J Neurochem 74, 490-500 (2000).
17. Goedert, M., Spillantini, M. G., Potier, M. C., Ulrich, J. & Crowther, R. A. Cloning and sequencing of the cDNA encoding an isoform of microtubule-associated protein tau containing four tandem repeats: differential expression of tau protein mRNAs in human brain. EMBO J 8, 393-399 (1989).
18. Andreadis, A. Tau gene alternative splicing: expression patterns, regulation and modulation of function in normal brain and neurodegenerative diseases. Biochim Biophys Acta 1739, 91-103 (2005).
19. Hutton, M. et al. Association of missense and 5′-splice-site mutations in tau with the inherited dementia FTDP-17. Nature 393, 702-705 (1998).
20. Spillantini, M. G. et al. Mutation in the tau gene in familial multiple system tauopathy with presenile dementia. Proc Natl Acad Sci USA 95, 7737-7741 (1998).
21. Goedert, M., Crowther, R. A. & Spillantini, M. G. Tau mutations cause frontotemporal dementias. Neuron 21, 955-958 (1998).
22. Spillantini, M. G. & Goedert, M. Tau pathology and neurodegeneration. Lancet Neurol 12, 609-622 (2013).
23. Lister, R. et al. Global epigenomic reconfiguration during mammalian brain development. Science 341, 1237905 (2013).
24. Peng, X. et al. Tissue-specific transcriptome sequencing analysis expands the non-human primate reference transcriptome resource (NHPRTR). Nucleic Acids Res 43, D737-742 (2015).
25. Rohlfs, R. V. & Nielsen, R. Phylogenetic ANOVA: The expression variance and evolution model for quantitative trait evolution. Syst Biol 64, 695-708 (2015).
26. Hansen, T. F. Stabilizing selection and the comparative analysis of adaptation. Evolution 51, 1341-1351 (1997).
27. Butler, M. A. & King, A. A. Phylogenetic comparative analysis: a modeling approach for adaptive evolution. Am. Nat. 164, 683-695 (2004).
28. Youn, Y. H. & Han, Y. G. Primary cilia in brain development and diseases. Am J Pathol 188, 11-22 (2018).
29. Spassky, N. et al. Primary cilia are required for cerebellar development and Shh-dependent expansion of progenitor pool. Dev Biol 317, 246-259 (2008).
30. Wiegering, A., Ruther, U. & Gerhardt, C. The ciliary protein Rpgrip11 in development and disease. Dev Biol 442, 60-68 (2018).
31. Harrison, P. W. & Montgomery, S. H. Genetics of cerebellar and neocortical expansion in anthropoid primates: A comparative approach. Brain Behav Evol 89, 274-285 (2017).
32. van der Lee, R., Wiel, L., van Dam, T. J. P. & Huynen, M. A. Genome-scale detection of positive selection in nine primates predicts human-virus evolutionary conflicts. Nucleic Acids Res 45, 10634-10648 (2017).
33. Moreira, A. et al. Hearing sensitivity of primates: recurrent and episodic positive selection in hair cells and stereocilia protein-coding genes. Genome Biol Evol (2021).
34. Grover, A. et al. 5′ splice site mutations in tau associated with the inherited dementia FTDP-17 affect a stem-loop structure that regulates alternative splicing of exon 10. J Biol Chem 274, 15134-15143 (1999).
35. Yeo, G. & Burge, C. B. Maximum entropy modeling of short sequence motifs with applications to RNA splicing signals. J Comput Biol 11, 377-394 (2004).
36. Jaganathan, K. et al. Predicting splicing from primary sequence with deep learning. Cell 176, 535-548 e524 (2019).
37. Zody, M. C. et al. Evolutionary toggling of the MAPT 17q21.31 inversion region. Nat Genet 40, 1076-1083 (2008).
38. Stefansson, H. et al. A common inversion under selection in Europeans. Nat Genet 37, 129-137 (2005).
39. Chang, C. W., Shao, E. & Mucke, L. Tau: Enabler of diverse brain disorders and target of rapidly evolving therapeutic strategies. Science 371 (2021).
40. Wang, Y. & Mandelkow, E. Tau in physiology and pathology. Nat Rev Neurosci 17, 5-21 (2016).
41. Kosik, K. S., Orecchio, L. D., Bakalis, S. & Neve, R. L. Developmentally regulated expression of specific tau sequences. Neuron 2, 1389-1397 (1989).
42. Sharma, G. et al. Tau isoform expression and phosphorylation in marmoset brains. J Biol Chem 294, 11433-11444 (2019).
43. Holzer, M., Craxton, M., Jakes, R., Arendt, T. & Goedert, M. Tau gene (MAPT) sequence variation among primates. Gene 341, 313-322 (2004).
44. Ingram, E. M. & Spillantini, M. G. Tau gene mutations: dissecting the pathogenesis of FTDP-17. Trends Mol Med 8, 555-562 (2002).
45. Poorkaj Tau is a candidate gene for chromosome 17 frontotemporal dementia. Annals of Neurology 44, 428-428 (1998).
46. D'Souza, I. et al. Missense and silent tau gene mutations cause frontotemporal dementia with parkinsonism-chromosome 17 type, by affecting multiple alternative RNA splicing regulatory elements. Proc Natl Acad Sci USA 96, 5598-5603 (1999).
47. D'Souza, I. & Schellenberg, G. D. Determinants of 4-repeat tau expression. Coordination between enhancing and inhibitory splicing sequences for exon 10 inclusion. J Biol Chem 275, 17700-17709 (2000).
48. D'Souza, I. & Schellenberg, G. D. tau Exon 10 expression involves a bipartite intron 10 regulatory sequence and weak 5′ and 3′ splice sites. J Biol Chem 277, 26587-26599 (2002).
49. Charizanis, K. et al. Muscleblind-like 2-mediated alternative splicing in the developing brain and dysregulation in myotonic dystrophy. Neuron 75, 437-450 (2012).
50. Goodwin, M. et al. MBNL sequestration by toxic RNAs and RNA misprocessing in the myotonic dystrophy brain. Cell Rep 12, 1159-1168 (2015).
51. Weyn-Vanhentenryck, S. M. et al. Precise temporal regulation of alternative splicing during neural development. Nat Commun, 2189 (2018).
52. Gorath, M., Stahnke, T., Mronga, T., Goldbaum, O. & Richter-Landsberg, C. Developmental changes of tau protein and mRNA in cultured rat brain oligodendrocytes. Glia 36, 89-101 (2001).
53. Zhang, C., Lee, K.-Y., Swanson, M. S. & Darnell, R. B. Prediction of clustered RNA-binding protein motif sites in the mammalian genome. Nucleic Acids Res 41, 6793-6807 (2013).
54. Du, H. et al. Aberrant alternative splicing and extracellular matrix gene expression in mouse models of myotonic dystrophy. Nat Struct Mol Biol 17, 187-193 (2010).
55. Goers, E. S., Purcell, J., Voelker, R. B., Gates, D. P. & Berglund, J. A. MBNL1 binds GC motifs embedded in pyrimidines to regulate alternative splicing. Nucleic Acids Res. 38, 2467-2484 (2010).
56. Kalbfuss, B., Mabon, S. A. & Misteli, T. Correction of alternative splicing of tau in frontotemporal dementia and parkinsonism linked to chromosome 17. J Biol Chem 276, 42986-42993 (2001).
57. Konermann, S. et al. Transcriptome engineering with RNA-targeting type VI-D CRISPR effectors. Cell 173, 665-676 e614 (2018).
58. Stefanoska, K. et al. An N-terminal motif unique to primate tau enables differential protein-protein interactions. J Biol Chem 293, 3710-3719 (2018).
59. Shaw-Smith, C. et al. Microdeletion encompassing MAPT at chromosome 17q21.3 is associated with developmental delay and learning disability. Nat Genet 38, 1032-1037 (2006).
60. Koolen, D. A. et al. A new chromosome 17q21.31 microdeletion syndrome associated with a common inversion polymorphism. Nat Genet 38, 999-1001 (2006).
61. Caffrey, T. M. & Wade-Martins, R. Functional MAPT haplotypes: bridging the gap between genotype and neuropathology. Neurobiol Dis 27, 1-10 (2007).
62. Saito, T. et al. Humanization of the entire murine Mapt gene provides a murine model of pathological human tau propagation. J Biol Chem 294, 12754-12765 (2019).
63. Geula, C. et al. Aging renders the brain vulnerable to amyloid beta-protein neurotoxicity. Nat Med 4, 827-831 (1998).
64. Brawand, D. et al. The evolution of gene expression levels in mammalian organs. Nature 478, 343-348 (2011).
65. Yan, Q. et al. Systematic discovery of regulated and conserved alternative exons in the mammalian brain reveals NMD modulating chromatin regulators. Proc Natl Acad Sci USA 112, 3445-3450 (2015).
66. Oba, S. et al. A Bayesian missing value estimation method for gene expression profile data. Bioinformatics 19, 2088-2096 (2003).
67. Li, M. et al. Integrative functional genomic analysis of human brain development and neuropsychiatric risks. Science 362 (2018).
68. Fairbrother, W. G., Yeh, R.-F., Sharp, P. A. & Burge, C. B. Predictive identification of exonic splicing enhancers in human genes. Science 297, 1007-1013 (2002).
69. Wang, Z. F. et al. Systematic identification and analysis of exonic splicing silencers. Cell 119, 831-845 (2004).
70. Zhang, X. H.-F. & Chasin, L. A. Computational definition of sequence motifs governing constitutive exon splicing. Genes Dev. 18, 1241-1250 (2004).
71. Zhang, C., Li, W.-H., Krainer, A. R. & Zhang, M. Q. RNA landscape of evolution for optimal exon and intron discrimination. Proc. Natl. Acad. Sci. USA 105, 5797-5802 (2008).
72. Sznajder, L. J. et al. Loss of MBNL1 induces RNA misprocessing in the thymus and peripheral blood. Nat Commun 11, 2022 (2020).
73. Feng, H. et al. Modeling RNA-binding protein specificity in vivo by precisely registering protein-RNA crosslink sites. Mol Cell 74, 1189-1204 e1186 (2019).
74. Benzing, T. & Schermer, B. Transition zone proteins and cilia dynamics. Nat Genet 43, 723-724 (2011).
75. Barak, M. et al. Human iPSC-Derived Neural Models for Studying Alzheimer's Disease: from Neural Stem Cells to Cerebral Organoids. Stem Cell Rev Rep 18, 792-820 (2022).
76. Wray, S. Modeling tau pathology in human stem cell derived neurons. Brain Pathol 27, 525-529 (2017).
77. Sposito, T. et al. Developmental regulation of tau splicing is disrupted in stem cell-derived neurons from frontotemporal dementia patients with the 10+16 splice-site mutation in MAPT. Hum Mol Genet 24, 5260-5269 (2015).
78. Ehrlich, M. et al. Distinct Neurodegenerative Changes in an Induced Pluripotent Stem Cell Model of Frontotemporal Dementia Linked to Mutant TAU Protein. Stem Cell Reports 5, 83-96 (2015).

It is to be understood that the agents and methods for treating or preventing a neurodegenerative disease by administering a treating agent that targets at least one muscleblind-like protein (MBNL) binding site near exon 10 of microtubule-associated protein tau (MAPT) are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A composition for treating a neurodegenerative disease, the composition comprising at least one component that targets at least one muscleblind-like protein (MBNL) binding site near exon 10 of microtubule-associated protein tau (MAPT) to block MBNL binding thereto and modulate exon 10 splicing.

2. The composition according to claim 1, wherein the at least one component targets at least two MBNL binding sites.

3. The composition according to claim 2, wherein the at least one component targets two MBNL binding sites.

4. The composition according to claim 1, wherein the at least one component comprises nuclease-inactive dCas13d in complex with one or more guide RNAs (gRNAs).

5. The composition according to claim 1, wherein the at least one component comprises one or more antisense nucleotides (ASOs).

6. The composition of claim 5, wherein the composition is useful for treating frontal temporal dementia (FTD).

7. The composition of claim 5, wherein the one or more ASOs comprise one or more of ASO 1 and ASO 4.

8. The composition of claim 6, wherein the one or more ASOs comprise one or more of ASO 1 and ASO 4.

9. A method of treating a neurodegenerative disease comprising the step of administering to a patient in need thereof an effective dose of a pharmaceutical composition which targets at least one muscleblind-like protein (MBNL) binding site near exon 10 of microtubule-associated protein tau (MAPT) to block MBNL binding thereto and modulate exon 10 splicing.

10. The method of treating a neurodegenerative disease as recited in claim 9, wherein the neurodegenerative disease is frontal temporal dementia (FTD).

11. The method of treating a neurodegenerative disease as recited in claim 9, wherein the at least one MBNL binding site near exon 10 is at least two binding sites.

12. The method of treating a neurodegenerative disease as recited in claim 10, wherein the at least one MBNL binding site near exon 10 is at least two binding sites.

13. The method of treating a neurodegenerative disease as recited in claim 11, wherein the at least one MBNL binding site near exon 10 is two binding sites.

14. The method of treating a neurodegenerative disease as recited in claim 12, wherein the at least one MBNL binding site near exon 10 is two binding sites.

15. The method of treating a neurodegenerative disease as recited in claim 9, wherein the pharmaceutical composition comprises nuclease-inactive dCas13d in complex with one or more guide RNAs (gRNAs).

16. The method of treating a neurodegenerative disease as recited in claim 9, wherein the pharmaceutical composition comprises one or more antisense oligo nucleotides (ASOs).

17. A method of expressing adult MAPT splice isoforms in human cells, the method comprising over expressing MBNL proteins in the human cells.

18. The method of claim 17, wherein the human cells are HEK293T cells.

19. A method of modeling tauopathies using human cells, the method comprising the method of expressing adult MAPT splice isoforms as recited in claim 17.

20. A method of modeling tauopathies using human cells, the method comprising the method of expressing adult MAPT splice isoforms as recited in claim 18.
